SEQUENCE LISTING (87 sequences)

SEQ ID NO: 1 - protein, 7 aa, artificial
  heavy chain, CDR1
GFTFSTF

SEQ ID NO: 2 - protein, 10 aa, artificial
  heavy chain, CDR1
GFTFSTFGMH

SEQ ID NO: 3 - protein, 5 aa, artificial
  heavy chain, CDR1
TFGMH

SEQ ID NO: 4 - protein, 6 aa, artificial
  heavy chain, CDR2
SGGSST

SEQ ID NO: 5 - protein, 10 aa, artificial
  heavy chain, CDR2
YISGGSSTIY

SEQ ID NO: 6 - protein, 17 aa, artificial
  heavy chain, CDR2
YISGGSSTIYYADTVKG

SEQ ID NO: 7 - protein, 8 aa, artificial
  heavy chain, CDR3
GYPYGLDY

SEQ ID NO: 8 - protein, 11 aa, artificial
  light chain, CDR1
KASQDINWYLS

SEQ ID NO: 9 - protein, 7 aa, artificial
  light chain, CDR2
RGNRLVD

SEQ ID NO: 10 - protein, 9 aa, artificial
  light chain, CDR3
LQYDEFPFT

SEQ ID NO: 11 - protein, 7 aa, artificial
  light chain, CDR2
RGNRLVE

SEQ ID NO: 12 - protein, 7 aa, artificial
  heavy chain, CDR1
GYSFTSY

SEQ ID NO: 13 - protein, 10 aa, artificial
  heavy chain, CDR1
GYSFTSYWMN

SEQ ID NO: 14 - protein, 5 aa, artificial
  heavy chain, CDR1
SYWMN

SEQ ID NO: 15 - protein, 6 aa, artificial
  heavy chain, CDR2
HPSDSE

SEQ ID NO: 16 - protein, 10 aa, artificial
  heavy chain, CDR2
MIHPSDSETR

SEQ ID NO: 17 - protein, 17 aa, artificial
  heavy chain, CDR2
MIHPSDSETRLSQKFKD

SEQ ID NO: 18 - protein, 9 aa, artificial
  heavy chain, CDR3
FDWDRAMDY

SEQ ID NO: 19 - protein, 11 aa, artificial
  light chain, CDR1
RASENIFSYLA

SEQ ID NO: 20 - protein, 7 aa, artificial
  light chain, CDR2
NTRSLAE

SEQ ID NO: 21 - protein, 9 aa, artificial
  light chain, CDR3
QHHYGTPWT

SEQ ID NO: 22 - protein, 7 aa, artificial
  heavy chain, CDR1
GYTFSEY

SEQ ID NO: 23 - protein, 10 aa, artificial
  heavy chain, CDR1
GYTFSEYTMH

SEQ ID NO: 24 - protein, 5 aa, artificial
  heavy chain, CDR1
EYTMH

SEQ ID NO: 25 - protein, 6 aa, artificial
  heavy chain, CDR2
NPNNGG

SEQ ID NO: 26 - protein, 10 aa, artificial
  heavy chain, CDR2
SINPNNGGTT

SEQ ID NO: 27 - protein, 17 aa, artificial
  heavy chain, CDR2
SINPNNGGTTYNQKFKG

SEQ ID NO: 28 - protein, 17 aa, artificial
  heavy chain, CDR2
SINPNQGGTTYNQKFKG

SEQ ID NO: 29 - protein, 13 aa, artificial
  heavy chain, CDR3
TRDYDNDGGLFAY

SEQ ID NO: 30 - protein, 11 aa, artificial
  light chain, CDR1
RASKNISKYLA

SEQ ID NO: 31 - protein, 11 aa, artificial
  light chain, CDR1
RASKNILKYLA

SEQ ID NO: 32 - protein, 7 aa, artificial
  light chain, CDR2
SGSTLQS

SEQ ID NO: 33 - protein, 9 aa, artificial
  light chain, CDR3
QQHYEYPFT

SEQ ID NO: 34 - protein, 7 aa, artificial
  heavy chain, CDR1
GFNIKDT

SEQ ID NO: 35 - protein, 10 aa, artificial
  heavy chain, CDR1
GFNIKDTYMH

SEQ ID NO: 36 - protein, 5 aa, artificial
  heavy chain, CDR1
DTYMH

SEQ ID NO: 37 - protein, 6 aa, artificial
  heavy chain, CDR2
DPASGN

SEQ ID NO: 38 - protein, 10 aa, artificial
  heavy chain, CDR2
KIDPASGNTK

SEQ ID NO: 39 - protein, 17 aa, artificial
  heavy chain, CDR2
KIDPASGNTKYDPQFQG

SEQ ID NO: 40 - protein, 10 aa, artificial
  heavy chain, CDR3
PYGNDYAMNY

SEQ ID NO: 41 - protein, 11 aa, artificial
  light chain, CDR1
RASEKIYSFLA

SEQ ID NO: 42 - protein, 7 aa, artificial
  light chain, CDR2
NAETLAE

SEQ ID NO: 43 - protein, 9 aa, artificial
  light chain, CDR3
QHHYGTPYT

SEQ ID NO: 44 - protein, 117 aa, artificial
  heavy chain variable region
DVQLVESGGGLVQPGGSRKLSCAASGFTFSTFGMHWVRQAPEKGLEWVAYISGGSSTIYYADTVKGRFTISRDNPKNTLFLQMTSLRSEDTAMYYCASGYPYGLDYWGQGTSVTVSS

SEQ ID NO: 45 - DNA, 351 bp, artificial
  heavy chain variable region
gatgtgcagctggtggagtctgggggaggcttagtgcagcctggagggtcccggaaactctcctgtgcagcctctggattcactttcagtacctttggaatgcactgggttcgtcaggctccagagaaggggctggagtgggtcgcatacattagtggtggcagtagtaccatctactatgcagacacagtgaagggccgattcaccatctccagagacaatcccaagaacaccctgttcctgcaaatgaccagtctaaggtctgaggacacggccatgtattactgtgcaagcggctacccctatggtttggactactggggtcaaggaacctcagtcaccgtctcctca

SEQ ID NO: 46 - protein, 107 aa, artificial
  light chain variable region
DIDMTQSPSSMYASLGERVTITCKASQDINWYLSWFQQKPGKSPKTLIYRGNRLVDGVPSRFSGSGSGQDYSLTISSLEYEDMGIYYCLQYDEFPFTFGSGTKLEIK

SEQ ID NO: 47 - DNA, 321 bp, artificial
  light chain variable region
gacatcgacatgacccagtctccatcttccatgtatgcatctctaggagagagagtcactatcacttgcaaggcgagtcaggacattaattggtatttaagttggttccagcagaaaccagggaaatctcctaagaccctgatctatcgtggaaacagattggtagatggggtcccatcaaggttcagtggcagtggatctgggcaagattattctctcaccatcagcagcctggagtatgaagatatgggaatttattattgtctacagtatgatgagtttccattcacgttcggctcggggacaaagttggaaataaaa

SEQ ID NO: 48 - protein, 117 aa, artificial
  heavy chain variable region
EVQLVESGGGLVQPGGSLRLSCAASGFTFSTFGMHWVRQAPGKGLEWVSYISGGSSTIYYADTVKGRFTISRDNAKNSLYLQMNSLRAEDTAVYYCASGYPYGLDYWGQGTLVTVSS

SEQ ID NO: 49 - DNA, 351 bp, artificial
  heavy chain variable region
gaggtgcagctggtggagagcggggggggactggtgcagccaggaggaagcctgagactgagctgtgccgcaagcgggttcacatttagtacctttggaatgcactgggtgaggcaggcccccggcaaagggctggagtgggtgtcttatatttccggcggaagtagcaccatatactacgctgatacagtgaagggcagattcaccataagcagggacaacgccaagaacagcctgtacctgcagatgaacagcctgagagccgaagacaccgctgtgtactactgcgccagcggctacccctacggcctggattactggggacaaggaacactggtgaccgtgagcagc

SEQ ID NO: 50 - protein, 107 aa, artificial
  light chain variable region
DIQMTQSPSSLSASVGDRVTITCKASQDINWYLSWFQQKPGKAPKTLIYRGNRLVEGVPSRFSGSGSGQDYTFTISSLQPEDMATYYCLQYDEFPFTFGQGTKVEIK

SEQ ID NO: 51 - DNA, 321 bp, artificial
  light chain variable region
gacattcagatgacccagagccccagcagcctgagcgccagcgtgggagacagagtgaccataacctgcaaagccagccaagacatcaactggtatctgtcctggtttcagcagaagcccggcaaagccccaaaaaccctcatctaccggggaaacagactggtggaaggggtgcccagcagatttagcggaagcggcagcggccaggactacaccttcaccatatcaagcctgcagcccgaagacatggccacctactactgcctgcagtacgacgaatttccatttaccttcggacagggaaccaaagtcgagatcaaa

SEQ ID NO: 52 - protein, 118 aa, artificial
  heavy chain variable region
QVQLQQPGAELVRPGASVKLSCKASGYSFTSYWMNWVKQRPGQGLEWIGMIHPSDSETRLSQKFKDKATLTVDKSSSTAYMQLSSPTSEDSAVYYCTRFDWDRAMDYWGQGTSVTVSS

SEQ ID NO: 53 - DNA, 354 bp, artificial
  heavy chain variable region
caggtccaactgcagcagcctggggctgagctggtgaggcctggagcttcagtgaagctgtcctgcaaggcttctggctactccttcaccagctactggatgaactgggtgaagcagaggcctggacaaggccttgagtggattggcatgattcatccttccgatagtgaaactaggttaagtcagaagttcaaggacaaggccacattgactgtagacaaatcctccagcacagcctacatgcaactcagcagcccgacatctgaggactctgcggtctattactgtacaagattcgactgggaccgggctatggactactggggtcaaggaacctcagtcaccgtctcctca

SEQ ID NO: 54 - protein, 107 aa, artificial
  light chain variable region
DIQMTQSPASLSASVGETVTITCRASENIFSYLAWYQQKQGKSPQLLVYNTRSLAEGVPSRFSGSGSGTQFSLKINSLQPEDFGTYYCQHHYGTPWTFGGGTKLEIK

SEQ ID NO: 55 - DNA, 321 bp, artificial
  light chain variable region
gacatccagatgactcagtctccagcctccctatctgcatctgtgggagaaactgtcaccatcacatgtcgagcaagtgagaatattttcagttatttagcatggtatcaacagaaacagggaaaatctcctcagctcctggtctataatacaagatccttagcagaaggtgtgccatcaaggttcagtggcagtggatcaggcacacagttttctctgaagatcaacagcctgcagcctgaagattttgggacttattactgtcaacatcattatggtactccgtggacgttcggtggaggcaccaagctggaaatcaaa

SEQ ID NO: 56 - protein, 118 aa, artificial
  heavy chain variable region
QVQLVQSGAEVKKPGASVKVSCKASGYSFTSYWMNWVRQAPGQGLEWMGMIHPSDSETRLSQKFKDRVTMTRDTSTSTVYMELSSLRSEDTAVYYCARFDWDRAMDYWGQGTLVTVSS

SEQ ID NO: 57 - DNA, 354 bp, artificial
  heavy chain variable region
caggtccaactcgtccaaagcggcgcagaagtcaaaaagcccggcgcatcagtcaaagttagctgcaaggccagcggctacagcttcacatcatactggatgaactgggtgcggcaagcccccggccaaggtctcgaatggatgggaatgatccaccccagcgacagcgagaccaggctgagccagaaatttaaagacagagtcaccatgaccagagacacctccacctcaaccgtctatatggaactgagcagcctcagaagcgaggacaccgccgtgtattactgcgcccggttcgactgggacagagccatggactactggggccaaggcaccctcgttaccgtgagcagc

SEQ ID NO: 58 - protein, 107 aa, artificial
  light chain variable region
DIQMTQSPSSLSASVGDRVTITCRASENIFSYLAWYQQKPGKAPKLLIYNTRSLAEGVPSRFSGSGSGTDFTLTISSLQPEDFATYYCQHHYGTPWTFGQGTKLEIK

SEQ ID NO: 59 - DNA, 321 bp, artificial
  light chain variable region
gacattcagatgacccagagccccagcagcctgagcgccagcgtgggagacagagtgaccataacctgcagagccagcgagaacatattctcatacctcgcctggtaccagcagaaacccggcaaagccccaaaactgctcatctacaacacaagaagcctggctgaaggagtgcccagcagattcagcgggtcaggcagcggcaccgacttcaccctgaccatcagcagcctgcaaccagaagacttcgccacctactactgccaacaccactacggcaccccctggaccttcggacaaggcaccaaactcgagatcaaa

SEQ ID NO: 60 - protein, 122 aa, artificial
  heavy chain variable region
EVHLQQSGPELMKPGASVKISCKTSGYTFSEYTMHWVKQSHGKSLEWIGSINPNNGGTTYNQKFKGKATLTVDKSSSTAYMELRSLTSEDSAVYNCARTRDYDNDGGLFAYWGQGTLVTVSA

SEQ ID NO: 61 - DNA, 366 bp, artificial
  heavy chain variable region
gaggtccacctgcaacagtctggacctgagctgatgaagcctggggcttcagtgaagatatcctgcaagacttctggatacacattcagtgaatacaccatgcactgggtgaagcagagccatggaaagagccttgagtggattggaagtattaatcctaacaatggtggtactacctacaaccagaagttcaagggcaaggccacattgactgtagacaagtcctccagcacagcctacatggagctccgcagcctgacatctgaggattctgcagtctataactgtgcaagaactagggactatgataacgacgggggtctttttgcttactggggccaagggactctggtcactgtctctgca

SEQ ID NO: 62 - protein, 107 aa, artificial
  light chain variable region
DVQITQSPSYLAASPGETITINCRASKNISKYLAWYQEKPGKTNKLLIYSGSTLQSGIPSRFSGNRSGTDFTLTISSLEPEDFAMYYCQQHYEYPFTFGGGTKLEIK

SEQ ID NO: 63 - DNA, 321 bp, artificial
  light chain variable region
gatgtccagataacccagtctccatcttatcttgctgcatctcctggagaaaccattactattaattgcagggcaagtaagaacattagcaaatatttagcctggtatcaagagaaacctgggaaaactaataagcttcttatctactctggatccactttgcaatctggaattccatcaaggttcagtggcaatagatctggtacagatttcactctcaccatcagtagcctggagcctgaagattttgcaatgtattactgtcaacaacattatgaatacccgttcacgttcggaggggggaccaagctggaaataaaa

SEQ ID NO: 64 - protein, 122 aa, artificial
  heavy chain variable region
QVQLVQSGAEVKKPGASVKVSCKTSGYTFSEYTMHWVRQAPGQRLEWMGSINPNQGGTTYNQKFKGRVTITVDKSASTAYMELSSLRSEDTAVYYCARTRDYDNDGGLFAYWGQGTLVTVSS

SEQ ID NO: 65 - DNA, 366 bp, artificial
  heavy chain variable region
caggtccaactcgtccaaagcggcgcagaagtcaaaaagcccggcgcatcagtcaaagttagctgcaagacatccggctacaccttcagcgagtacaccatgcactgggtgagacaagccccaggccaaagactggagtggatgggaagcatcaaccccaaccaaggcggcaccacctacaaccaaaaattcaagggcagagtgacaattaccgtggacaagagcgccagcaccgcctacatggagctgtctagcctgagaagcgaggacaccgccgtgtactattgcgccagaaccagagactacgacaacgatggaggactgttcgcctattggggccagggaaccctcgtgaccgtgagcagc

SEQ ID NO: 66 - protein, 107 aa, artificial
  light chain variable region
DVQITQSPSFLSASVGDRVTITCRASKNILKYLAWYQQKPGKAPKLLIYSGSTLQSGVPSRFSGSRSGTEFTLTISSLQPEDFATYYCQQHYEYPFTFGQGTKLEIK

SEQ ID NO: 67 - DNA, 321 bp, artificial
  light chain variable region
gacgtgcagattacccaaagccccagcttcctgtccgccagcgtgggcgacagagtgacaattacatgcagagccagcaagaacatactgaagtacctggcatggtaccaacaaaaacccggcaaggcccccaaactgctcatctactccggcagtaccctgcagagcggcgtgcccagcagattcagcggaagcagaagcggcaccgagttcactctgaccatcagcagcctccaaccagaggacttcgccacctactactgccagcagcactacgaataccccttcaccttcggccagggcaccaagctggagatcaaa

SEQ ID NO: 68 - protein, 119 aa, artificial
  heavy chain variable region
EVQLQQSGAELVKPGASVTLSCTVSGFNIKDTYMHWVKQRPEQGLEWIGKIDPASGNTKYDPQFQGKATITADTSSNTAYLHLSSLTSEDSAVYFCASPYGNDYAMNYWGQGTSVTVSS

SEQ ID NO: 69 - DNA, 357 bp, artificial
  heavy chain variable region
gaggttcagctgcagcagtctggggcagaacttgtgaagccaggggcctcagtcacgttgtcctgcacagtttctggcttcaacattaaagacacctatatgcactgggtgaaacagaggcctgaacagggcctggagtggattggaaagattgatcctgcgagtggtaatactaaatatgacccgcagttccagggcaaggccactataacagcagacacatcctccaacacagcctacctgcacctcagcagcctgacatctgaggacagtgccgtctatttctgtgctagcccctatggtaacgactatgctatgaactactggggacaaggaacctcagtcaccgtctcctca

SEQ ID NO: 70 - protein, 107 aa, artificial
  light chain variable region
DIQMTQSPASLSASVGETVTITCRASEKIYSFLAWYQQKQEKSPQLLVYNAETLAEGVPSRFSGTGSGIQFSLKIISLQPEDFGIYYCQHHYGTPYTFGGGTKLEIK

SEQ ID NO: 71 - DNA, 321 bp, artificial
  light chain variable region
gacatccagatgactcagtctccagcctccctatctgcatctgtgggagaaactgtcaccatcacatgtcgagcaagtgagaaaatttacagttttttagcatggtatcagcagaaacaggaaaaatctcctcaactcctggtctataatgcagaaaccttagcagaaggtgtgccatcaaggttcagtggcactggatcgggcatccagttttctctgaagattatcagcctgcagcctgaagattttgggatttattactgtcaacatcattatggtactccgtacacgttcggaggggggaccaagttggaaataaaa

SEQ ID NO: 72 - protein, 119 aa, artificial
  heavy chain variable region
EVQLVQSGAEVKKPGATVKISCKVSGFNIKDTYMHWVQQAPGKGLEWMGKIDPASGNTKYDPQFQGRVTITADTSTNTAYMELSSLRSEDTAVYYCATPYGNDYAMNYWGQGTLVTVSS

SEQ ID NO: 73 - DNA, 357 bp, artificial
  heavy chain variable region
gaggtgcagctggtgcagagcggagcagaggtgaagaagccaggggccacagtgaagataagctgtaaggtgagcggattcaacattaaggacacatatatgcactgggtgcagcaggcacccggcaaaggactggagtggatgggaaagatcgaccccgccagtggcaataccaagtacgacccccagttccagggccgagtgaccatcaccgcagacaccagcaccaacacagcctacatggagctgagcagcctccgcagcgaagacacagccgtgtactactgcgccaccccctatggcaacgactacgctatgaattactggggccagggaacactggtcaccgtgtccagc

SEQ ID NO: 74 - protein, 107 aa, artificial
  light chain variable region
DIQMTQSPSSLSASVGDRVTITCRASEKIYSFLAWYQQKPGKAPKLLLYNAETLAEGVPSRFSGSGSGIDFTLTISSLQPEDFATYYCQHHYGTPYTFGGGTKVEIK

SEQ ID NO: 75 - DNA, 321 bp, artificial
  light chain variable region
gacattcagatgacccagagccccagcagcctgagcgccagcgtgggagacagagtgaccataacctgcagagccagcgagaaaatatatagctttctggcctggtatcagcagaagcccggaaaagccccaaaactgctgctgtacaacgcagaaaccctggcagagggagtgcccagcagattcagcggatcaggaagcggcatcgacttcaccctgaccatcagcagcctgcaacccgaagacttcgccacctactactgccaacaccactacggcaccccatacaccttcggaggaggcacaaaggtcgaaatcaaa

SEQ ID NO: 76 - protein, 107 aa, artificial
  light chain variable region
DIQMTQSPSSLSASVGDRVTITCKASQDINWYLSWFQQKPGKAPKTLIYRGNRLVDGVPSRFSGSGSGQDYTFTISSLQPEDMATYYCLQYDEFPFTFGQGTKVEIK

SEQ ID NO: 77 - DNA, 321 bp, artificial
  light chain variable region
gacattcagatgacccagagccccagcagcctgagcgccagcgtgggagacagagtgaccataacctgcaaagccagccaagacatcaactggtatctgtcctggtttcagcagaagcccggcaaagccccaaaaaccctcatctaccggggaaacagactggtggacggagtgcccagcagattcagcggaagcggcagcgggcaagactacaccttcaccatatcaagcctgcagcccgaagacatggccacctactactgcctgcagtacgacgaattcccctttaccttcggccaagggaccaaggtggagatcaag

SEQ ID NO: 78 - protein, 294 aa, Staphylococcus aureus
MADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 79 - DNA, 891 bp, Staphylococcus aureus
catatggcagacagcgacattaacattaaaaccggcaccaccgatatcggaagcaataccaccgtaaaaaccggagatctggtcacctatgataaagaaaacggcatgctgaaaaaggtgttttatagctttatcgacgataaaaaccacaataaaaagctgttagtgatccgcaccaaaggtacaatcgcaggtcaatacagagtttatagcgaagaaggtgccaataaaagcggtctggcatggccgagcgcatttaaagtgcagctgcagctgccggacaatgaagttgcacagataagcgactattatccacgtaatagtattgacacaaaggaatatatgagcaccctgacctatggttttaacggtaatgttaccggtgatgacaccggtaagattgggggattaattggtgcaaatgtttctattggtcacaccctgaaatatgttcaaccggattttaagaccatcctggaatctccgacagataaaaaggtggggtggaaagttatctttaataatatggttaaccagaactggggcccgtatgatcgcgatagctggaatccagtttatggtaatcagctgttcatgaaaacacgcaatggcagcatgaaagcagcagataactttctggacccgaataaagcaagcagcctgctgagcagcggtttttcaccggactttgcaaccgttatcaccatggatcgcaaagcctcaaaacagcagaccaacattgatgttatctacgaaagagtacgcgatgattaccagttacattggacaagcaccaattggaaagggaccaataccaaagacaaatggaccgatagaagcagcgaacggtataaaattgattgggagaaagaagaaatgaccaatctcgag

SEQ ID NO: 80 - protein, 294 aa, artificial
  alpha-hemolysin mutant
MADSDINIKTGTTDIGSNTTVKTGDLVTYDKENGMLKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 81 - DNA, 891 bp, artificial; identical to SEQ ID NO: 79
  alpha-hemolysin mutant

SEQ ID NO: 82 - protein, 129 aa, artificial
  heavy chain variable region
EVQMVQSGAEVKKPGEPLKISCKGSGYKFGTHWIGWVRQRPGKGLEWMGIIHPADSETKYSPSFQGQVSFSADKSSNTAYLHWSTLRASDTAMYYCARRSGSSSWYALDFWGQGTMVTVSSASTKGPSV

SEQ ID NO: 83 - protein, 123 aa, artificial
  light chain variable region
QSVLTQSPSASGTPGQRVTISCSGGSSNIGSNTVNWYQQFPGAAPKLLIYTNNQRPSGVPDRFSGSKSGTSASLAISGLQSEDEADYYCATWDDSLNGLYVFGTGTKVTVLGQPKANPTVTLF

SEQ ID NO: 84 - protein, 122 aa, artificial
  heavy chain variable region
EVQLVESGGGLVQPGGSLRLSCAASGFTFSSHDMHWVRQATGKGLEWVSGIGTAGDTYYPDSVKGRFTISRENAKNSLYLQMNSLRAGDTAVYYCARDRYSPTGHYYGMDVWGQGTTVTVSS

SEQ ID NO: 85 - protein, 106 aa, artificial
  light chain variable region
DIQMTQSPSTLSASVGDRVTITCRASQSISSWLAWYQQKPGKAPKLLIYKASSLESGVPSRFSGSGSGTEFTLTISSLQPDDFATYYCKQYADYWTFGQGTKVEIK

SEQ ID NO: 86 - protein, 330 aa, artificial [sequence omitted]

SEQ ID NO: 87 - protein, 107 aa, artificial [sequence omitted]

(19) United States
(12) Patent Application Publication
(10) Pub. No.: US 2022/0324949 A1
(43) Pub. Date: Oct. 13, 2022

(54) ANTI-ALPHA-HEMOLYSIN ANTIBODY AND USE THEREOF

(71) Applicant: MABWELL (SHANGHAI) BIOSCIENCE CO., LTD., Shanghai (CN)

(72) Inventors: Maomao AN, Shanghai (CN); Lichun WANG, Shanghai (CN); Pan GAO, Shanghai (CN); Jian LIN, Shanghai (CN); Yuanying JIANG, Shanghai (CN); Hui SHEN, Shanghai (CN); Simin CHEN, Shanghai (CN); Shiyu GUO, Shanghai (CN); Wei FANG, Shanghai (CN)

(21) Appl. No.: 17/761,959

(22) PCT Filed: Sep. 18, 2020
(86) PCT No.: PCT/CN2020/116184

§ 371 Date: Mar. 18, 2022

(30) Foreign Application Priority Data
Sep. 20, 2019 (CN) 201910889697.5

(51) Int. Cl. C07K 16/12; A61P 31/04

(52) U.S. Cl. CPC C07K 16/1271; A61P 31/04; A61K 2039/505

(57) ABSTRACT

Provided are an antibody that binds to Staphylococcus aureus alpha-hemolysin or a fragment thereof, and the use of the antibody or the fragment thereof for preventing or treating Staphylococcus aureus infections. The antibody is obtained through screening by means of the strategies of attenuated immunity and virulent screening of alpha-hemolysin, and the antibody has high affinity to alpha-hemolysin, can effectively block the hemolysis effect of alpha-hemolysin, has proved significant protective or therapeutic effects in the alpha-hemolysin sepsis model, MRSA bacteremia model and MRSA lung infection model, and has a synergistic effect with antibiotics, which is a beneficial supplement to existing antibiotic therapy for Staphylococcus aureus.

The present application claims the priority benefit of Chinese Patent Application No. CN201910889697.5 filed on Sep. 20, 2019, which is hereby incorporated by reference in its entirety.

### TECHNICAL FIELD

The present invention relates to the field of antibody drugs, in particular to an anti-alpha-hemolysin antibody and pharmaceutical application thereof.

### BACKGROUND OF THE PRESENT INVENTION

Staphylococcus aureus, belonging to Staphylococcus, is an important gram-positive pathogenic bacterium, and is the most important G+ pathogenic bacterium in human. Staphylococcus aureus can cause local infections, such as pyogenic infection, pneumonia, pseudomembranous enteritis, pericarditis, etc., as well as systemic infections, such as septicemia, sepsis, etc. Data available on the China Antimicrobial Surveillance Network showed Staphylococcus aureus ranked 4th among pathogenic bacteria detected in hospitals and 1st among G+ bacteria detected in hospitals.

Staphylococcus aureus releases a large amount of toxins to destroy tissue cells during a process of infection in human body, and inhibits immune cells in the body from eliminating pathogens. At present, beta-lactam antibiotics are mainly adopted clinically to treat Staphylococcus aureus infection. However, antibiotics only inhibit or kill bacteria, but do nothing to the toxins released by bacteria. On the contrary, bacteria release more toxins under the pressure of antibiotics, and bacteria killed by antibiotics release toxins upon lysis also. When a large amount of toxins enter blood, the host immune system will be excessively activated to release excessive inflammatory factors, thereby forming sepsis.

Moreover, in the process of fighting with human beings, Staphylococcus aureus has become less and less sensitive to beta-lactam antibiotics, for example, methicillin-resistant Staphylococcus aureus (MRSA) is separated more and more at present. Data available on the China Antimicrobial Surveillance Network showed in 2016 the isolating rate of MRSA was even as high as 38.9%. For MRSA, therapeutic drugs clinically mainly include a limited number of ones, for example, vancomycin and linezolid, which however are typical representatives of drugs that can result in “super-resistant” bacteria infecting humans. Therefore, clinical MRSA infection becomes increasing serious, and the selection of antibacterial agents is very limited, both of which lead to clinical treatment failure in patients and a high mortality rate of patients. Even infection caused by VRSA (vancomycin-resistant Staphylococcus aureus) resistant to vancomycin has been cropping up clinically in recent years, and humans are about to face the situation of no cure.

Studies found that pathogenic factors released by Staphylococcus aureus include hemolysin, leukocidin, enterotoxin, coagulase, and the like. Hemolysin is one of major virulence factors secreted by Staphylococcus aureus and can be divided into four types, alpha-hemolysin, beta-hemolysin, gamma-hemolysin and delta-hemolysin. Alpha-hemolysin (Hla) is a secreted toxin protein encoded by gene HLA of Staphylococcus aureus, expressed in almost all strains. Compared with other types of hemolysin, alpha-hemolysin is a more critical virulence factor affecting pathogenicity of Staphylococcus aureus, and researches showed that Staphylococcus aureus with alpha-hemolysin-expressing gene deleted has a significantly reduced toxicity in infecting animals. Alpha-hemolysin protein has a full length of 319 amino acids and a relative molecular weight of 33 kD, and is secreted in a monomer form. As one of the members of pore forming toxin family, the most well-defined biological property of alpha-hemolysin is the ability to rapidly lyse red blood cells and other tissue cells in host. Alpha-hemolysin binds to cholesterol and sphingomyelin on host cells, and then they aggregate together to form a heptamer with a relative molecular weight of about 232 kD, which in turn folds to form a β-barrel transmembrane structure with a diameter of about 1.5 nm, allowing intracellular water, ions and other small molecules to leak and thereby causing hemolysis or death of target cells. In addition, alpha-hemolysin can also cause the contraction and spasm of smooth muscles of capillary vessels, thereby resulting in the blockage of the capillary vessels and causing ischemia and tissue necrosis. So it plays an important role in the processes that Staphylococcus aureus causes diseases such as sepsis, pneumonia, mammary gland infection, cornea infection, serious skin infection and the like. At the same time, alpha-hemolysin also destroys leukocytes in infected tissues of a host, prevents the host from clearing Staphylococcus aureus infected. Under the pressure of host immune system and antibiotics, Staphylococcus aureus at infected areas releases a great amount of alpha-hemolysin, and when the released alpha-hemolysin enters blood, the host immune system will be activated to release excessive inflammatory factors, thereby forming sepsis.

In case of the continuous emergence of superbacteria, antibody drugs have become a powerful tool of the post-antibiotic era. Antibody is one kind of natural protein produced by adaptive immune system, and a human body is administered with antibody drugs through passive immunization, so that the antibody not only can neutralize virulence factors, but also can strengthen the immune response capability of a host to pathogens, and can accelerate the elimination of infected pathogens. Alpha-hemolysin shows its potential as an anti-infection antibody drug target for the treatment of Staphylococcus aureus infection for the following reasons.

Alpha-hemolysin is highly conserved among Staphylococcus aureus strains and is expressed in almost all Staphylococcus aureus strains; alpha-hemolysin has definite biological functions and plays an important role in the process of forming host infection and developing sepsis by Staphylococcus aureus; and no homologous protein of alpha-hemolysin exists in mammalian cells, so an antibody drug can be designed by targeting alpha-hemolysin, with low potential off-target possibility and low toxic and side effects.

Therefore, alpha-hemolysin is an ideal target for an antibody drug against Staphylococcus aureus infection, and an effective neutralization of alpha-hemolysin is beneficial to blocking Staphylococcus aureus infection in a human body, avoiding occurrence of sepsis after serious infection and improving prognosis of a clinically infected patient. Presently, fully human antibody R-301 (Salvecin®) developed by Aridis Pharmaceuticals, Inc. targeting alpha-hemolysin has entered phase III clinical trial, and main indications are severe pneumonia caused by Staphylococcus aureus (including methicillin-resistant Staphylococcus aureus); humanized antibody MEDI4893 developed by Astrazeneca Pharmaceuticals Inc. targeting alpha-hemolysin is under phase II clinical investigation, and is mainly used for prevention and treatment of Staphylococcus aureus (including methicillin-resistant Staphylococcus aureus) pneumonia.

Currently antibiotics such as vancomycin and the like are still the first line drugs for treating Staphylococcus aureus infection, but in view of the continuous increase in drug resistance and the slow development of new antibiotics, there is still a need in the art to develop novel and highly effective antibody drugs against Staphylococcus aureus by directly neutralizing toxins. Due to the hemolytic activity of Staphylococcus aureus alpha hemolysin, preparation strategies of corresponding monoclonal antibodies mainly include: using an alpha hemolysin-based toxoid as an immunogen to prepare a monoclonal antibody by hybridoma technology; screening an in vitro antibody library by using alpha-hemolysin as a target protein; and the like. These methods have difficulty in obtaining an anti-alpha-hemolysin antibody having high affinity and high biological activity for virulent alpha-hemolysin. Regeneron Pharmaceuticals Inc. adopted an attenuated alpha-hemolysin as immunogen, and detected affinities of positive clones through surface plasma resonance, which however had a low screening efficiency and could not efficiently obtain antibodies with high biological activity.

### SUMMARY OF THE PRESENT INVENTION

The technical problem to be solved by the present invention to improve traditional preparation method of anti-alpha-hemolysin antibody molecules, and following strategies are utilized in the present invention: solving the virulence problem of alpha-hemolysin in animals through attenuated toxicity immunization, ensuring the high affinity of an antibody to wild-type virulent alpha-hemolysin through strong toxicity screening, ensuring the biological activity of a monoclonal antibody from the very beginning through erythrocyte lysis inhibition assay, thereby improving the screening efficiency of anti-alpha-hemolysin therapeutic monoclonal antibodies. Further, the technical problem to be solved by the present invention is to provide an anti-alpha-hemolysin antibody molecule, in particular a humanized anti-alpha-hemolysin monoclonal antibody, which has the ability to bind to Staphylococcus aureus alpha hemolysin and inhibit the hemolysin from lysing blood cells and damaging tissue cells, and can be used alone or in combination with existing anti-bacterial agents fur the treatment of infection or infection-related diseases caused by alpha-hemolysin or alpha-hemolysin producing microorganisms.

In view of the above problems, the present invention is directed to provide an antibody or a functional fragment thereof, and uses thereof based on the antibody or the functional fragment thereof.

Technical solutions of the present invention are as follows.

In one aspect, the present invention provides a method for preparing an anti-alpha-hemolysin monoclonal antibody, including steps as follows:

(1) immunizing animals with an attenuated alpha-hemolysin or an alpha-hemolysin without virulence;

(2) taking splenocytes and preparing hybridomas;

(3) screening an antibody having high affinity to virulent alpha-hemolysin or wild-type alpha-hemolysin using the same;

(4) selecting an antibody which has an activity of inhibiting erythrocyte lysis caused by virulent alpha-hemolysin or wild-type alpha-hemolysin through an assay of inhibition of erythrocyte lysis.

The assay of inhibition of erythrocyte lysis includes mixing a diluted antibody (or a hybridoma supernatant) with an equal volume of alpha-hemolysin, adding erythrocytes in for incubation, centrifuging and obtaining a supernatant, and detecting the inhibitory activity of the antibody on erythrocyte lysis by alpha-hemolysin according to OD450.

Preferably, in the method for preparing an anti-alpha-hemolysin monoclonal antibody according to the present invention, the virulent alpha-hemolysin or wild-type alpha-hemolysin is Staphylococcus aureus alpha-hemolysin, and preferably has an amino acid sequence as shown in SEQ ID NO: 78; and the attenuated alpha-hemolysin or alpha-hemolysin without virulence is alpha-hemolysin with H35L, and preferably has an amino acid sequence as shown in SEQ ID NO: 80.

Preferably, in the method for preparing an anti-alpha-hemolysin monoclonal antibody according to the present invention, the virulent alpha-hemolysin or wild-type alpha-hemolysin, the attenuated alpha-hemolysin, or the alpha-hemolysin without virulence is independently linked to a purification tag, a carrier protein, and/or an adjuvant molecule; and the linkage includes coupling, cross-linking, conjugation, and/or fusion.

In another aspect, the present invention also provides an anti-alpha-hemolysin monoclonal antibody prepared by any one of the previous methods, the antibody having at least one activity selected from the group consisting of blocking hemolytic effect of alpha-hemolysin, blocking damage of alpha-hemolysin to lung epithelial cells, reducing bacterial load in MRSA infected lung tissue, prolonging survival time of a patient suffering from MRSA bacteremia, and preventing or alleviating sepsis caused by alpha-hemolysin.

The anti-alpha-hemolysin monoclonal antibody prepared by any one of the previous methods according to the present invention has the following structures:

VH-CDR1: selected from the group consisting of SEQ NOs: 1, 2, 3, 12, 13 14, 22, 23, 24, 34, 35, and 36;

VH-CDR2: selected from the group consisting of SEQ ID NOs: 4, 5, 6, 15, 16, 17, 25, 26, 27, 28, 37, 38, and 39;

VH-CDR3: selected from the group consisting of SEQ ID NOs: 7, 18, 29, and 40;

VL-CDR1: selected from the group consisting of SEQ ID NOs: 8, 19, 30, 31, and 41;

VL-CDR2: selected from the group consisting of SEQ ID NOs: 9, 11, 20, 32, and 42; and

VL-CDR3: selected from the group consisting of SEQ ID NOs: 10, 21, 33, and 43.

In a further aspect, the present invention provides an antibody or fragment thereof comprising a heavy chain variable region (VH) and a light chain variable region (VL), wherein the heavy chain variable region (VH) and the light chain variable region (VL) comprise a combination of CDRs (VH-CDR1, VH-CDR2, VH-CDR3; and VL-CDR1, VL-CDR2, VL-CDR3) selected from the group consisting of:

(1) VH-CDR1 as shown in SEQ ID NO: 1, VH-CDR2 as shown in SEQ ID NO: 4, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;

(2) VH-CDR1 as shown in SEQ ID NO: 2, VH-CDR2 as shown in SEQ ID NO: 5, and VH-CDR3 as shown in SEQ ID NO: 7; and, VL-CDRI as shown in SEQ ID NO: 8, VL-CDR2 as shown in SEQ ID NO: 9, and VL-CDR3 as shown in SEQ ID NO: 10;

(3) VH-CDR1 as shown in SEQ ID NO: 3, VH-CDR2 as shown in SEQ ID NO: 6, and VH-CDR3 as shown in SEQ ID NO: 7; and, VL-CDR1 as shown in SEQ ID NO: 8; VL-CDR2 as shown in SEQ ID NO: 9, and VL-CDR3 as shown in SEQ ID NO: 10;

(4) VH-CDR1 as shown in SEQ ID NO: 2, VH-CDR2 as shown in SEQ ID NO: 6, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;

(5) VH-CDR1 as shown in SEQ ID NO: 2, VH-CDR2 as shown in SEQ ID NO: 6, and VH-CDR3 as shown in SEQ ID NO: 7; and, VL-CDR1 as shown in SEQ ID NO: 8 VL-CDR2 as shown in SEQ ID NO: 11, and VL-CDR3 as shown in SEQ ID NO: 10;

(6) VH-CDR1 as shown in SEQ ID NO: 12, VH-CDR2 as shown in SEQ ID NO: 15, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;

(7) VH-CDR1 as shown in SEQ ID NO: 13, VH-CDR2 as shown in SEQ ID NO: 16, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;

(8) VH-CDR1 as shown in SEQ ID NO: 14, VH-CDR2 as shown in SEQ ID NO: 17, and VH-CDR3 as shown in SEQ ID NO: 18; and, VL-CDRI as shown in SEQ ID NO: 19, VL-CDR2 as shown in SEQ ID NO: 20, and VL-CDR3 as shown in SEQ ID NO: 21;

(9) VH-CDR1 as shown in SEQ ID NO: 13, VH-CDR2 as shown in SEQ ID NO: 17, and

VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;

(10) VH-CDR1 as shown in SEQ ID NO: 22, VH-CDR2 as shown in SEQ ID NO: 25, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;

(11) VH-CDR1 as shown in SEQ ID NO: 23, VH-CDR2 as shown in SEQ ID NO: 26, and VH-CDR3 as shown in SEQ ID NO: 29; and, VL-CDR1 as shown in SEQ ID NO: 30, VH-CDR2 as shown in SEQ ID NO: 32, and VL-CDR3 as shown in SEQ ID NO: 33;

(12) VH-CDR1 as shown in SEQ ID NO: 24, VH-CDR2 as shown in SEQ ID NO: 27, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;

(13) VH-CDR1 as shown in SEQ ID NO: 23, VH-CDR2 as shown in SEQ ID NO: 27, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;

(14) VH-CDR1 as shown in SEQ ID NO: 23, VH-CDR2 as shown in SEQ ID NO: 28, and VH-CDR3 as shown in SEQ ID NO: 29; and, VL-CDR1 as shown in SEQ ID NO: 31, VL-CDR2 as shown in SEQ ID NO: 32, and VL-CDR3 as shown in SEQ ID NO: 33;

(15) VH-CDR1 as shown in SEQ ID NO: 34, VH-CDR2 as shown in SEQ ID NO: 37, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43;

(16) VH-CDR1 as shown in SEQ ID NO: 35, VH-CDR2 as shown in SEQ ID NO: 38, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43;

(17) VH-CDR1 as shown in SEQ ID NO: 36, VH-CDR2 as shown in SEQ ID NO: 39, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43; and

(18) VH-CDR1 as shown in SEQ ID NO: 35, VH-CDR2 as shown in SEQ ID NO: 39, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43,

Preferably, in the antibody or fragment thereof provided by the present invention, the heavy chain variable region comprises:

an amino acid sequence as shown in any one of SEQ ID NO: 44, SEQ ID NO: 48, SEQ ID NO: 52, SEQ ID NO: 56, SEQ ID NO: 60, SEQ ID NO: 64, SEQ ID NO: 68 and SEQ ID NO: 72, or an amino acid sequence having at least 75% identity to the amino acid sequence as shown; and/or

the light chain variable region comprises:

an amino acid sequence as shown in any one of SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74 and SEQ ID NO: 76 or an amino acid sequence having at least 75% identity to the amino acid sequence as shown.

According to a particular embodiment of the present invention, the antibody or fragment thereof comprises a heavy chain variable region and a light chain variable region selected from the group consisting of following combinations:

(1) an amino acid sequence as shown in SEQ ID NO: 44 or an amino acid sequence having at least 75% identity to the amino acid sequence as shown in SEQ ID NO: 44; and, an amino acid sequence as shown in SEQ ID NO: 46 or an amino acid sequence having at least 75% identity to the amino acid sequence as shown in SEQ ID NO: 46;

(2) an amino acid sequence as shown in SEQ ID NO: 48 or an amino acid sequence haying at least 75% identity to the amino acid sequence as shown in SEQ ID NO: 48; and, an amino acid sequence as shown in SEQ ID NO: 50 or an amino acid sequence having at least 75% identity to the amino acid sequence as shown in SEQ ID NO: 50;

(3) an amino acid sequence as shown in SEQ ID NO: 52 or an amino acid sequence haying at least 75% identity to the amino acid sequence as shown in SEQ ID NO: 52; and, an amino acid sequence as shown in SEQ ID NO: 54 or an amino acid sequence having at least 75% identity to the amino acid sequence as shown in SEQ ID NO: 54;

(4) an amino acid sequence as shown in SEQ ID NO: 56 or an amino acid sequence having at least 75% identity to the amino acid sequence as shown in SEQ ID NO: 56; and, an amino acid sequence as shown in SEQ ID NO: 58 or an amino acid sequence having at least 75% identity to the amino acid sequence as shown in SEQ ID NO: 58;

(5) an amino acid sequence as shown in SEQ ID NO: 60 or an amino acid sequence having at least 75% identity to the amino acid sequence as shown in SEQ ID NO: 60; and, an amino acid sequence as shown in SEQ ID NO: 62 or an amino acid sequence having at least 75% identity to the amino acid sequence as shown in SEQ ID NO: 62;

(6) an amino acid sequence as shown in SEQ ID NO: 64 or an amino acid sequence having at least 75% identity to the amino acid sequence as shown in SEQ ID NO: 64; and, an amino acid sequence as shown in SEQ ID NO: 66 or an amino acid sequence having at least 75% identity to the amino acid sequence as shown in SEQ ID NO: 66;

(7) an amino acid sequence as shown in SEQ ID NO: 68 or an amino acid sequence having at least 75% identity to the amino acid sequence as shown in SEQ ID NO: 68; and, an amino acid sequence as shown in SEQ ID NO: 70 or an amino acid sequence having at least 75% identity to the amino acid sequence as shown in SEQ ID NO: 70;

(8) an amino acid sequence as shown in SEQ ID NO: 72 or an amino acid sequence having at least 75% identity to the amino acid sequence as shown in SEQ ID NO: 72; and, an amino acid sequence as shown in SEQ ID NO: 74 or an amino acid sequence having at least 75% identity to the amino acid sequence as shown in SEQ ID NO: 74; and

(9) an amino acid sequence as shown in SEQ ID NO: 48 or an amino acid sequence having at least 75% identity to the amino acid sequence as shown in SEQ ID NO: 48; and, an amino acid sequence as shown in SEQ ID NO: 76 or an amino acid sequence having at least 75% identity to the amino acid sequence as shown in SEQ ID NO: 76.

The at least 75% identity described above is any percent identity greater than or equal to 75%, such as at least 80%, preferably at least 85%, more preferably at least 90%, even more preferably at least 91%, 92%, 93%, 94%, 95%, 96%, 97%, 98%, or even 99% identity.

The antibody or fragment thereof provided by the present invention can be in any form, for example, a monoclonal antibody, a single-chain antibody, a single-domain antibody, a bifunctional antibody, a nanobody, a fully or partially humanized antibody or a chimeric antibody and the like; alternatively, the antibody or fragment thereof is a half-antibody or an antigen-binding fragment of the half-antibody, for example, single-chain variable fragment (scFv), bivalent single-chain variable fragment (BsFv), disulfide-stabilized Fv fragment (dsFv), (disulfide-stabilized. Fv fragment)₂ (dsFv)₂, antigen-binding fragment (Fab), Fab′ fragment, F(ab′)₂ fragment, or variable fragment (Fv); and with respect to the fragment of the antibody provided by the present invention, preferably, the fragment is any fragment of the antibody capable of specifically binding to the antigen Staphylococcus aureus alpha-hemolysin.

Alternatively, the antibody according to the present invention is IgA, IgD, IgG or IgM, more preferably IgG1. An fragment of the antibody is selected from the group consisting of say, Fab, F(ab′)₂ and Fv fragments of the antibody.

Preferably, the antibody or fragment thereof further comprises a human or murine constant region, preferably a human or murine light chain constant region (CL) and/or heavy chain constant region (CH); more preferably, the antibody or fragment thereof comprises a heavy chain constant region selected from the group consisting of IgG, IgA, IgM, IgD and IgE and/or a kappa or lambda type light chain constant region. According to a particular embodiment of the present invention, the antibody is a monoclonal antibody, preferably murine derived, chimeric or humanized monoclonal antibody; more preferably, the heavy chain constant region of the monoclonal antibody is of IgG1 or IgG4 subtype and the light chain constant region of the monoclonal antibody is of kappa type.

Preferably, the antibody or fragment thereof provided by the present invention comprises a heavy chain constant region as shown in SEQ NO: 86 and/or a light chain constant region as shown in SEQ ID NO: 87, or an amino acid sequence having at least 75% identity to either the heavy chain constant region or the light chain constant region as shown.

In still a further aspect, the present invention provides a nucleic acid molecule comprising a nucleotide sequence encoding any antibody or fragment thereof of the present invention, or encoding a heavy chain CDR, a light chain CDR, a heavy chain variable region, a light chain variable region, a heavy chain or a light chain comprised in the antibody or fragment thereof.

According to a particular embodiment of the present invention, the nucleic acid molecule comprises a nucleotide sequence encoding the heavy chain variable region or the light chain variable region of the antibody or fragment thereof according to the present invention. For example, the nucleic acid molecule comprises the nucleotide sequence as shown in any one of SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77.

In yet another aspect, the present invention provides a vector comprising the nucleic acid molecule according to the present invention. The vector can be an eukaryotic expression vector, a prokaryotic expression vector, an artificial chromosome, a phage vector, and the like.

The vector or nucleic acid molecule of the present invention may be used to transform or transfect a host cell or in any way enter a host cell for antibody preservation or expression, etc. Thus, in a further aspect, the present invention provides a host cell comprising the nucleic acid molecule and/or vector according to the present invention, or transformed or transfected with the nucleic acid molecule and/or vector according to the present invention. The host cell may be any prokaryotic or eukaryotic cell, such as a bacterial or insect, fungus, plant or animal cell.

According to the disclosure of the present invention, the antibody or fragment thereof, the nucleic acid molecule, the vector, and/or the host cell provided by the present invention can be Obtained using any conventional techniques known in the art. For example, with respect to an antibody, the heavy chain variable region and/or the light chain variable region of the antibody or the heavy chain and/or the light chain of the antibody may be obtained from the nucleic acid molecule provided by the present invention, and then the antibody is obtained by assembling them with optional other domains of the antibody; alternatively, the host cell provided by the present invention is cultured under conditions that allow the host cell to express the heavy chain variable region and/or the light chain variable region of the antibody or the heavy chain and/or the light chain of the antibody and assemble them into an antibody. Optionally, the method further includes a step of recovering the produced antibody.

The antibody or fragment thereof provided by the present invention may also be combined with other moieties, for example, a cell surface receptor, a small molecule compound such as amino acids and carbohydrates, a small molecule polymer, or any other moiety that modifies the antibody of the present invention, or even an active protein or polypeptide such as antimicrobial peptides or antibiotics. Thus, in another aspect, the present invention provides a conjugate or fusion protein comprising an antibody or fragment thereof provided by the present invention. For example, the conjugate or fusion protein can be a bispecific antibody comprising an antibody or fragment thereof according to the present invention.

The antibody or fragment thereof, the nucleic acid molecule, the vector, the host cell, the conjugate or fusion protein provided by the present invention may be contained in a pharmaceutical composition, more particularly, a pharmaceutical preparation, to be used for various purposes as actually needed. Thus, in a further aspect, the present invention also provides a pharmaceutical composition comprising an antibody or fragment thereof, a nucleic acid molecule, a vector, a host cell, a conjugate and/or a fusion protein according to the present invention, and optionally a pharmaceutically acceptable excipient.

For any purpose of use, the present invention also provides a kit comprising an antibody or fragment thereof, a nucleic acid molecule, a vector, a host cell, a conjugate, a fusion protein and/or a pharmaceutical composition of the present invention.

Based on their ability to bind to alpha-hemolysin and inhibit the hemolysin from lysing blood cells and damaging tissue cells, the antibody or fragment thereof according to the present invention can be used alone or in combination with any other antibacterial agent, to treat or ameliorate infection caused by alpha-hemolysin or alpha-hemolysin producing microorganisms, or other diseases or conditions resulting from the infection. Accordingly, the present invention also provides related applications of the subject matters described above.

In particular, in still a further aspect, the present invention provides use of an antibody or fragment thereof, a nucleic acid molecule, a vector, a host cell, a conjugate, a fusion protein and/or a pharmaceutical composition according to the present invention in the manufacture of a medicament for preventing or treating infection and complication caused by alpha-hemolysin or an alpha-hemolysin producing microorganism.

Furthermore, the present invention provides use of the antibody or fragment thereof, the nucleic acid molecule, the vectors, the host cell, the conjugate, the fusion protein and/or the pharmaceutical composition in combination with another antibacterial agent or anti-alpha-hemolysin antibody in the manufacture of a medicament for preventing or treating infection and complication caused by alpha-hemolysin or an alpha-hemolysin producing microorganism.

In addition, the present invention provides a method for preventing or treating infection and complication caused by alpha-hemolysin or an alpha-hemolysin producing microorganism, including administering to a subject in need thereof the antibody or fragment thereof, the nucleic acid molecule, the vector, the host cell, the conjugate, the fusion protein and/or the pharmaceutical composition, and optionally an antibacterial agent. The optional antibacterial agent may be an agent co-administered with the antibody or fragment thereof, the nucleic acid molecule, the vector, the host cell, the conjugate, the fusion protein and/or the pharmaceutical composition of the present invention. The co-administration of the two may be in any way, including simultaneously, sequentially or at intervals.

In the present invention, the alpha-hemolysin producing microorganism is preferably Staphylococcus aureus, including methicillin-resistant Staphylococcus aureus.

In the present invention, the infection caused by alpha-hemolysin or an alpha-hemolysin producing microorganism may be one or more selected from the group consisting of upper respiratory tract infection, pneumonia, severe pneumonia, abdominal infection, subcutaneous and soft tissue infection, bacteremia, and infection in various organs; and the complication may be one or more selected from the group consisting of Acute Respiratory Distress Syndrome (ARDS), sepsis, and a rise in inflammatory factors in body.

In the present invention, the another antibacterial agent is an agent (including chemical agents, biological agents and Chinese medicines) useful for the treatment and prevention of infection caused by Staphylococcus aureus, e.g. methicillin-resistant Staphylococcus aureus, preferably is an antibiotic, such as beta-lactam antibiotics. The antibiotic may be a drug listed in guidelines/treatment strategies issued by the Infectious Diseases Society of America (IDSA) or the Chinese Medical Association, preferably vancomycin, norvancomycin, teicoplanin, linezolid, daptomycin, cephapiprazole, fusidic acid, or ceftaroline.

The present invention also provides a method for diagnosing infection caused by alpha-hemolysin or an alpha-hemolysin producing microorganism, including contacting the antibody or fragment thereof, the nucleic acid molecule, the vector, the host cell, the conjugate, the fusion protein and/or the pharmaceutical composition with a sample from a subject.

In the present invention, a “subject” may be a mammal, such as a primate or rodent, more preferably a human.

In the present invention, terms “alpha-hemolysin” and “alpha-hemolysin toxin” interchangeably refer to a 33 kDa monomeric protein secreted by Staphylococcus aureus that oligomerizes into a heptameric structure in host cell membrane to form pores, resulting in cell lysis, inflammation, and tissue damage. The amino acid sequence of wild-type alpha-hemolysin is as shown in SEQ ID NO: 78. The amino acid sequence of modified alpha-hemolysin (H35L mutant) is as shown in SEQ ID NO: 80. Unless otherwise indicated, reference to alpha-hemolysin refers to the wild-type form. Hla-H35L is an alpha-hemolysin with histidine at position 35 changed to leucine. This allows the toxin to assemble on the host cell membrane till the pre-pore forms, but does not form a fully functional pore. This renders the alpha-hemolysin non-toxic.

In the present invention, term “antibody” is intended to refer to an immunoglobulin molecule comprised of four polypeptide chains, two heavy (H) chains and two light (L) chains interconnected by disulfide bonds (i.e., “full antibody molecule”), as well as a multimer thereof (e.g., IgM) or an antigen-binding fragment thereof. Each heavy chain comprises a heavy chain variable region (“HCVR” or “VH”) and a heavy chain constant region (comprising domains CH1, CH2 and CH3). Each light chain comprises a light chain variable region (“LCVR” or “VL”) and a light chain constant region (CL). The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. In certain embodiments of the present invention, the FRs of the antibody (or antigen binding fragment thereof) may be identical to human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in scientific literatures in which one or two CDRs can be dispensed with for binding. Padlan et al, (1995 FASEB J. 9: 133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320: 415-428). CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and ammo acids to substitute can also be selected empirically. Empirical substitutions can be conservative or non-conservative substitutions.

Term “specifically binds” or “binds specifically to”, or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less (e.g., a smaller KD denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, to bind specifically to alpha-hemolysin. Moreover, multi-specific antibodies that bind to one domain in alpha-hemolysin and one or more additional antigens or a bi-specific antibodies that bind to two different regions of alpha-hemolysin are nonetheless considered antibodies that “specifically bind”, as used herein.

Term “high affinity” antibody refers to those mAbs having a binding affinity to alpha-hemolysin, expressed as KD, of at least 10⁻⁷ M, preferably 10⁻⁸ M, more preferably 10⁻⁹ M, even more preferably 10⁻¹⁰ M, even more preferably 10⁻¹¹ M, as measured by surface plasmon resonance, BIACORE™ or solution-affinity ELISA.

Terms “slow off rate”, “K dissociation” and “kd” are intended to refer to an antibody that dissociates from alpha-hemolysin with a rate constant of 1×10⁻³ s⁻¹ or less, preferably 1×10⁻⁴ s⁻¹ or less, as determined by surface plasmon resonance, e.g., BIACORE™.

Terms “antigen-binding portion”, “antigen-binding fragment” of an antibody, and the like, as used herein, include any naturally occurring, enzymatically obtainable, synthetic, or genetically engineered polypeptide or glycoprotein that specifically binds an antigen to form a complex. Term “antigen-binding fragment” of an antibody or “antibody fragment”, as used herein, refers to one or more fragments of an antibody that retains the ability to bind to alpha-hemolysin.

In particular embodiments, the antibody or antibody fragment of the present invention may be conjugated to a therapeutic moiety (“immunoconjugate”), for example an antibiotic, a second anti-alpha-hemolysin antibody or an antibody directed against a cytokine such as IL-1, IL-6 or TGF-β or any other therapeutic moiety suitable for use in the treatment of a disease or condition including Staphylococcus aureus infection, skin and soft tissue infection (including abscess), surgical site infection, artificial joint infection, bacteremia, sepsis, septic arthritis, meningitis, osteomyelitis, endocarditis, pneumonia, toxic shock syndrome, mastitis and furuncle, and carbuncle (boil).

“Isolated antibody” as used herein, is intended to refer to an antibody that is substantially free of other antibodies (Abs) having different antigenic specificities (e.g., an isolated antibody that specifically binds alpha-hemolysin, or a fragment thereof, is substantially free of Abs that specifically bind antigens other than alpha-hemolysin).

Term “KD”, as used herein, is intended to refer to the equilibrium dissociation constant of a particular antibody-antigen interaction.

Compared with prior arts, Staphylococcus aureus alpha-hemolysin (alpha-Toxin) and a Staphylococcus aureus alpha-hemolysin mutant protein (alpha-Toxin H35L) without toxicity were obtained in the present invention by prokaryotic expression in Escherichia coli. Mice were successfully immunized with alpha-Toxin H35L and spleen cells were taken, and an antibody library was established using hybridoma technology. Anti-alpha-hemolysin monoclonal antibodies having high affinity to alpha-hemolysin (alpha-Toxin) and biological activity were obtained through library screening, and lead antibody molecules (of IgG1 subtype) were obtained after the anti-alpha-hemolysin monoclonal antibodies were further humanized.

Specifically, in the present invention mice were successfully immunized with alpha-Toxin H35L and spleen cells were taken, and an antibody library was established using hybridoma technology; anti-alpha-hemolysin monoclonal antibodies having high affinity to alpha-hemolysin (alpha-Toxin) and biological activity were obtained through library screening, and 16 lead antibody molecules in total were obtained, which not only have high affinity to alpha-hemolysin, but also have the activity of blocking hemolysis caused by alpha-hemolysin. Particularly, attenuated toxicity immunization and strong toxicity screening were adopted in the present invention with respect to antigen selection and antibody screening.

Human-mouse chimeric antibodies and humanized antibodies were recombinantly expressed based on the murine-derived lead antibody molecules, and finally humanized antibody molecules with biological activities similar to those of the murine-derived antibodies were obtained.

Pharmacodynamic studies in vitro showed the humanized antibodies of the present invention could, in a dose-dependent manner, block the hemolytic effect of alpha-hemolysin on rabbit erythrocytes and the damage effect of alpha-hemolysin on lung epithelial cells. In addition, in the present invention, an alpha-hemolysin-induced sepsis model, an MRSA bacteremia model and an MRSA lung infection model established in mice were utilized to carry out pharmacodynamic evaluation on the lead antibody molecules in animals. Research results showed that the humanized antibodies of the present invention had a remarkable protective effect on mice in the alpha-hemolysin-induced sepsis model; were capable of remarkably prolonging the survival time of mice in the MRSA bacteremia model; and were able to obviously reduce the bacterial load of tissues in mice in MRSA lung infection model. Moreover, the combined application of the humanized antibodies of the present application and conventional antibacterial agents such as vancomycin and linezolid, etc., showed a remarkable synergistic effect in the alpha-hemotysin-induced sepsis model, the MRSA bacteremia model and the MRSA lung infection model established in mice.

Furthermore, a pharmacokinetic study of a single dose of an antibody of the present invention was performed in cynomolgus monkeys at a dosage of 20 mg/kg (N=3), and the results showed that basic pharmacokinetic parameters met druggability criteria (Table 1): the in vivo elimination half-life (T1/2) was 137±36.9 h, and the plasma clearance (CL) was 0.501±0.222 ml/h/kg. In addition, an acute toxicity test research of an antibody of the present invention was carried out in mice (N=10), and the result showed that when a dosage of 125 mg/kg at the maximum was administered in 24 hours, no mice died, and no discomfort was shown by the animals during continuous observation for 14 days; no abnormality was seen in main organs (heart, liver, spleen, lung, kidney and brain) taken for histology observation from sacrificed animals; and when cynomolgus monkeys were administrated with a dosage of 10 mg/kg of an antibody of the present invention once, no death of the animals occurred, and no discomfort was shown by the animals during continuous observation for 28 days. Acute toxicity test research showed that the antibody of the present invention had good safety.

The antibody according to the present invention can effectively neutralize toxin, block damage of the toxin to tissue cells of a patient, and improve immunity of the patient, thereby relieving tissue injury due to clinical Staphylococcus aureus infection, promoting the infected bacteria in the body of the patient to be cleared more quickly, and preventing or relieving sepsis. Under the action of the antibody, patients can be more quickly converted from intravenous infusion treatment to oral treatment, and the treatment course can be shortened; meanwhile, the antibody according to the present invention has better clinical curative effect and tolerance, and is a favorable supplement for the existing antibiotic therapy.

### BRIEF DESCRIPTION OF THE DRAWINGS

Embodiments of the present invention are described in detail below with reference to the attached drawing figures, in which:

FIG. 1 shows the construction of a recombinant expression plasmid for His-tag fused Staphylococcus aureus alpha-hemolysin (alpha-Toxin) protein.

FIG. 2 shows the construction of a recombinant expression plasmid for His-tag fused Staphylococcus aureus alpha-hemolysin mutant (H35L alpha-Toxin) protein.

FIG. 3 shows the SDS-PAGE electrophoresis (10% gels) results of recombinantly expressed Staphylococcus aureus alpha-hemolysin and its mutant (H35L alpha-Toxin), in which panel 3A shows alpha-Toxin, and panel 3shows H35L alpha-Toxin, and the loading amounts are 10

FIG. 4 shows the hemolytic effect of Staphylococcus aureus alpha-hemolysin and its mutant (H35L alpha-Toxin) on sheep blood agar plates.

FIG. 5 shows the hemlolytic effect of Staphylococcus aureus alpha-hemolysin and its mutant (H35L alpha-Toxin) on rabbit blood, in which panel 5A shows alpha-Toxin and panel 5B shows H35L alpha-Toxin.

FIG. 6 shows the detection results obtained during screening of hybridorna cell strains, in which panel 6A shows the ELISA detection results of the binding of antibodies in the supernatants of different cell strains to alpha-hemolysin, and panel 6B shows the inhibition results of antibodies in the supernatants of different cell strains on alpha-hemolysin, respectively.

FIG. 7 shows the ELISA detection results of the binding of antibodies of the present invention to alpha-hemolysin, in which panels 7A to 7D show the binding of the screened antibodies 78D4, 16H4, 78F4 and 98G9 to alpha-hemolysin, respectively.

FIG. 8 shows the curves of association and dissociation as detected by Octct of antibodies of the present invention and alpha-hemolysin, in which panels 8A to 8D show the binding of the screened humanized versions of antibodies 78D4, 16H4, 78F4 and 98G9 to alpha-hemolysin, respectively.

FIG. 9 shows the effects of antibodies of the present invention on the hemolytic activity of alpha-hemolysin, in which panels 9A to 9C show the results at different antibody amounts, respectively.

FIG. 10 shows the therapeutic effects of antibodies of the present invention in animal model of alpha-hemolysin-induced sepsis.

FIG. 11 shows the therapeutic effects of antibodies of the present invention in animal model of methicillin-resistant Staphylococcus aureus-induced bacteremia.

FIG. 12 shows the therapeutic effects of antibodies of the present invention in animal model of methicillin-resistant Staphylococcus aureus pneumonia.

FIG. 13 shows the results of a pharmacokinetic study of antibody of the present invention after a single administration in cynomolgus monkeys.

### DETAILED DESCRIPTION OF PREFERRED EMBODIMENTS

The present invention is illustrated below with reference to specific examples. It will be understood by those skilled in the art that these examples are merely illustrative of the present invention and do not limit the scope of the present invention in any way.

Experimental procedures in the following examples are all conventional ones, unless otherwise specified. Raw materials and reagents used in the following examples are all commercially available products, unless otherwise specified.

Known antibodies as follows are used herein as controls:

Fully human antibody R-301 (Salvecin®), abbreviated as AR (see U.S. Pat. No. 9,249,215B2), from Aridis Pharmaceuticals, Inc., of which heavy chain variable region is as shown in SEQ ID NO: 82 and light chain variable region is as shown in SEQ ID NO: 83.

Humanized antibody MEDI4893, abbreviated as AZ (see US20140072577A1), from Astrazeneca Pharmaceuticals Inc., of which heavy chain variable region is as shown in SEQ ID NO: 84 and light chain variable region is as shown in SEQ ID NO: 85.

The antibodies provided by the present invention as follows have a heavy chain constant region as shown in SEQ ID NO: 86 and a light chain constant region as shown in SEQ ID NO: 87.

### EXAMPLE 1

### Recombinant Expression of His-Tag Fused Staphylococcus aureus Alpha-Hemolysin (Alpha-Toxin)

A nucleotide sequence corresponding to the amino acid sequence of Staphylococcus aureus alpha-hemolysin which was used as a target sequence was artificially synthesized, and was cloned into plasmid pET-21a containing His tag utilizing sites for restriction enzymes NdeI and XhoI. The amino acid sequence of Staphylococcus aureus alpha-hemolysin is as shown in SEQ ID NO: 78, the corresponding nucleotide sequence is as shown in SEQ ID NO: 79, and the construction of the recombinant plasmid is shown in FIG. 1.

The obtained recombinant plasmid was transformed into competent BL21(DE3) pLysS cells, and single colonies were picked up next day and inoculated into LB liquid medium containing 100 μg/mL ampicillin, followed by shaking culture at 37° C. overnight. The overnight culture was inoculated into LB liquid medium containing 100 μg/mL ampicillin in a volume ratio of 1:100, and a shaking culture was performed at 37° C. at 200 rpm till OD₆₀₀ of about 0.6-0.8. IPTG was added into the culture to reach a final concentration of 0.1 mM, and induction was performed at 16° C. for 16-18 h. After induction, the bacterial culture was taken and centrifuged at 8,000 rpm for 3 min, and bacterial cells were collected and preserved at −80° C.

### EXAMPLE 2

### Recombinant Expression of His-Tag Fused Staphylococcus aureus Alpha-Hemolysin Mutant (H35L Alpha-Toxin)

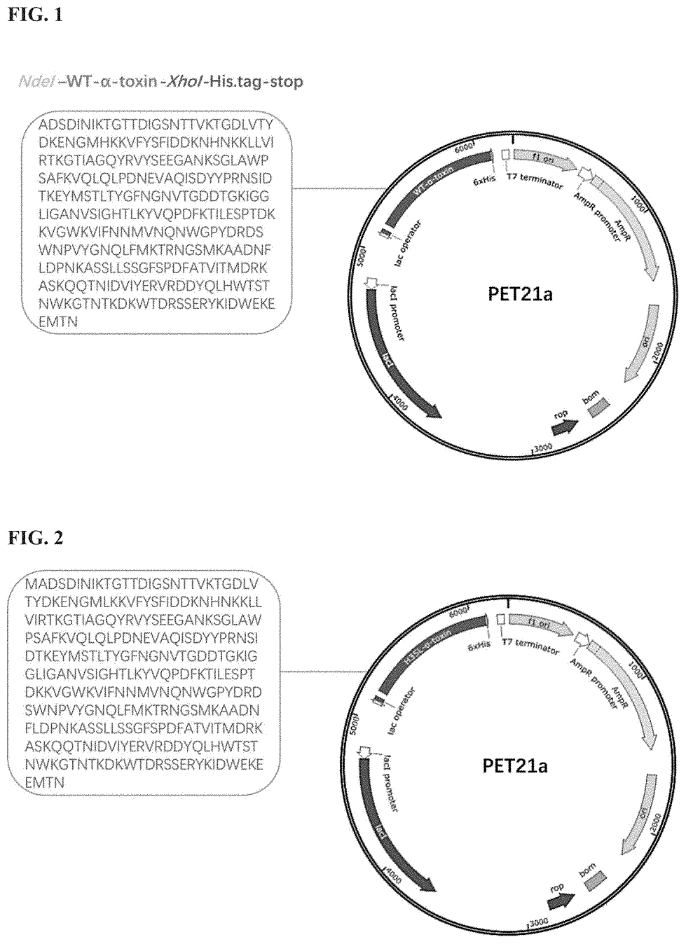

Based on the amino acid sequence of Staphylococcus aureus alpha-hemolysin, a mutated amino acid sequence was obtained by mutating histidine (His) at position 35 to leucine (Leu). A nucleotide sequence corresponding to the mutated amino acid sequence was artificially synthesized, and was cloned into plasmid Pet-21a containing His tag utilizing sites for restriction enzymes NdeI and XhoI. The amino acid sequence of Staphylococcus aureus alpha-hemolysin mutant (H35L alpha-Toxin) is as shown in SEQ ID NO: 80 (H35L is at position 36 in that sequence), the corresponding nucleotide sequence is as shown in SEQ ID NO: 81, and the construction of the recombinant plasmid is shown in FIG. 2.

The obtained recombinant plasmid was transformed into competent BL21(DE3) pLysS cells, and single colonies were picked up next day and inoculated into LB liquid medium containing 100 μg/mL ampicillin, followed by shaking culture at 37° C. overnight. The overnight culture was inoculated into LB liquid medium containing 100 μg/mL ampicillin in a volume ratio of 1:100, and a shaking culture was performed at 37° C. at 200 rpm till OD₆₀₀ of about 0.6-0.8. IPTG was added into the culture to reach a final concentration of 0.25 mM, and induction was performed at 25° C. for 4.5 h. After induction, the bacterial culture was taken and centrifuged at 8,000 rpm for 3 min, and bacterial cells were collected and preserved at −80° C.

### EXAMPLE 3

### Purification of Staphylococcus aureus Alpha-Hemolysin (Alpha-Toxin) and its Mutant (H35L Alpha-Toxin)

Escherichia coli cells with Staphylococcus aureus alpha-hemolysin and its mutant inducibly expressed therein were sonicated respectively with an ultrasonic system at 180 W with a mode of operating for 3 sec, and pausing for 3 sec, 7-9 min in total. After centrifugation at 13,000 rpm for 30 min, supernatant was collected and sterilized by filtration through a 0.22 μm filter.

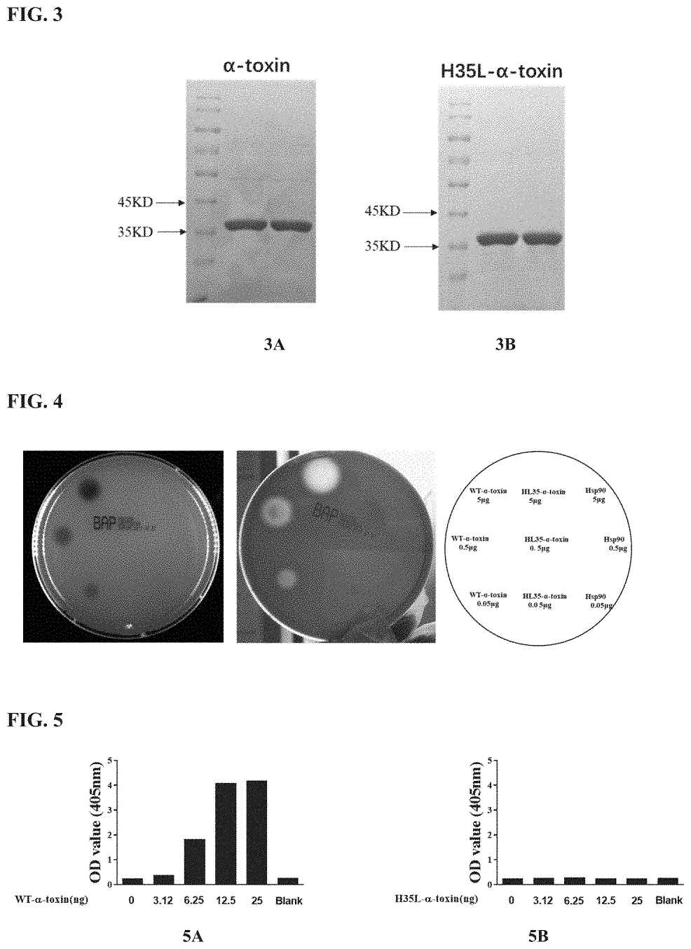

A Ni column was packed with filler and then was mixed with the filtered supernatant on a rotary mixer for 1 h at room temperature. The Ni column was firstly eluted with 5 column volumes of Buffer 1 (containing imidazole at a concentration of 30 mM) till the color of developing solution did not change, to remove proteins non-specifically bound to the column, and then was eluted with 5 column volumes of Buffer 2 (containing imidazole at a concentration of 300 mM) to obtain target proteins. Then, eluates containing target proteins were concentrated and displaced using a 10 KD concentrator in PBS as a solvent. The electrophoresis results of the obtained protein are shown in FIG. 3.

### EXAMPLE 4

### Detection of Biological Activity of Staphylococcus aureus Alpha-Hemolysin (Alpha-Toxin) and its Mutant (H35L Alpha-Toxin) (In Vitro Hemolysis Assay and In Vivo Toxicity Assay in Mice)

Respectively, 10 μL of each of Staphylococcus aureus alpha-hemolysin and its mutant in different concentrations (5 μg/mL, 0.5 μg/mL and 0.05 μg/mL) was dripped on the surface of a sheep blood agar plate (Shanghai Comagai Microbial Technology CO., LTD), and the plates were placed in an incubator at 37° C. for incubation for 24 h. Then hemolytic rings were obviously visible around the alpha-hemolysin dripped, having diameters related to concentrations of the alpha-hemolysin dripped; while no hemolytic ring was found around the alpha-hemolysin mutant dripped. The results of the assay are shown in FIG. 4.

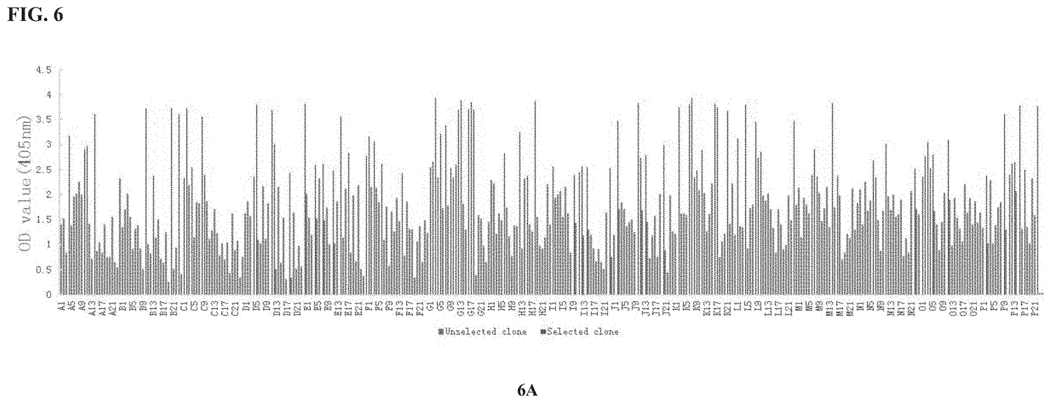

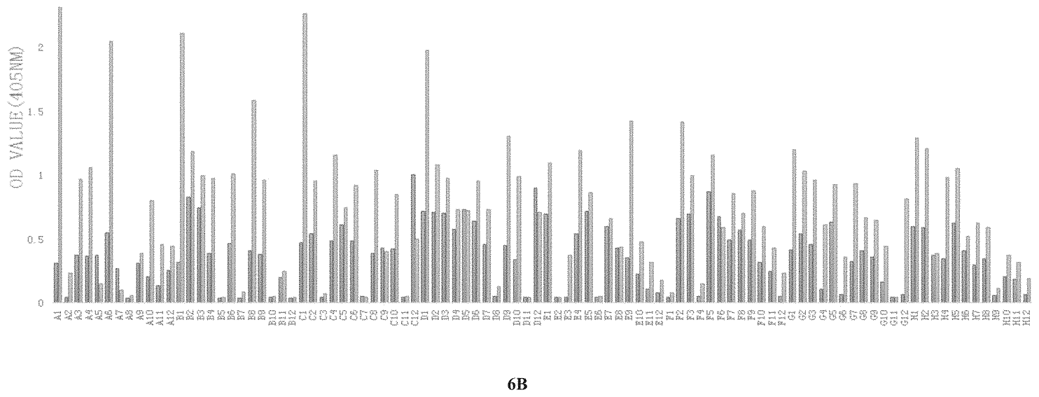

75 μL of 5% rabbit erythrocytes (Bio-channel Biotechnology) and different amounts of Staphylococcus aureus alpha-hemolysin or its mutant were added to the wells of a 96-well plate, and PBS was added to obtain a final volume of 150 μL. The 96-well plate was incubated in an incubator at 37° C. for 1 h, and then was centrifuged at 3000 rpm for 3 min. 100 μL of the supernatant from each well was evaluated for hemolytic activity by absorbance at 405 nm (OD405) measured using a microplate reader. The results showed that alpha-hemolysin had a dose-dependent hemolytic activity on rabbit erythrocytes, whereas the alpha-hemolysin mutant had no hemolytic activity, The results of the assay are shown in FIG. 5.

Different dosages of Staphylococcus aureus alpha-hemolysin or its mutant recombinantly expressed according to the methods described in Examples 1, 2 and 3 were administered to C57 mice by tail vein injection. It was found that the lowest lethal dosage of Staphylococcus aureus alpha-hemolysin in C57 mice was 3 μg/mouse; while no any discomfort was seen with the mice administered with the highest dosage 200 μg/mouse of the alpha-hemolysin mutant.

### EXAMPLE 5

### Immunization of C57 Mice With Staphylococcus aureus Alpha-Hemolysin Mutant (H35L Alpha-Toxin)

Alpha-hemolysin or alpha-hemolysin mutant was diluted to different concentrations with PBS and injected via tail vein into C57 mice. Toxic effects of the two on C57 mice were compared, to choose an immunogen and dosage thereof suitable for the subsequent immunization of animals. The results are shown in Table 1.

TABLE 1
Toxic effects of different dosages of alpha-hemolysin and
alpha-hemolysin mutant on mice

| Protein | Dosage | Performance of the mice |
|---|---|---|
| WT α-toxin | 50 μg/mouse (2.5 mg/kg) | Instantly died |
| | 10 μg/mouse (0.5 mg/kg) | Instantly died |
| | 3 μg/mouse (0.15 mg/kg) | Died within 30 min |
| | 1 μg/mouse (0.05 mg/kg) | Discomforts including tremble, fidget, and |
| | | the like exhibited; survived; and recovered |
| | | 16 hours later (overnight) |
| | 0.1 μg/mouse (0.005 mg/kg) | No obvious discomfort shown |
| H35L α-toxin | 200 μg/mouse (10 mg/kg) | No obvious discomfort shown |
| Unrelated | 200 μg/mouse (10 mg/kg) | No obvious discomfort shown |
| protein BSA | | |

### EXAMPLE 6

### Immunization of Balb/c Mice With Staphylococcus aureus Alpha-Hemolysin Mutant (H35L Alpha-Toxin)

8 week old Balblc mice were immunized using a procedure of 42 days in total at 14 day interval, with reference to Antibodies a Laboratory Manual, Second Edition (Edward A. Greenfield 2012).

The Staphylococcus aureus alpha-hemolysin mutant was emulsified in complete or incomplete Freund's adjuvant and injected into the subcutaneous tissue and peritoneal cavity of the nape of neck, tail root and groin of each mouse in a unilateral manner. On the 35th day of immunization, tail vein blood was collected, antibody titer was detected by ELISA, and spleen cells of the immunized mice were collected and fused with myeloma cells.

### EXAMPLE 7

### Screening and Identification of Hybridoma Cell Strains and Sequencing of Antibodies

Spleen cells of Balb/c mice immunized with Staphylococcus aureus alpha-hemolysin mutant (H35L alpha-Toxin) were taken and fused with myeloma cells P3X63Ag8.653 using PEG or electrofusion method. The fused hybridoma cells were inoculated in 30 384-well plates, and 24 hours later, HAT-containing medium and HT-containing medium were added to select hybridoma cells, After cultured in 384-well plates for 10-14 days, supernatants of cell cultures were taken and subjected to ELISA assay with α-Toxin to screen hybridoma clones capable of secreting antibodies that specifically bind to alpha-hemolysin (see panel 6A). Subsequently, OD values from the wells detected by ELISA were ordered from the highest to the lowest, and wells per plate with values in the top 94 were selected and cells therein were transferred to a 96-well plate for culture (among the 384-well plates, plate NO. 30 was less positive for ELISA detection, so no wells of the plate were selected for transferring); and 29 96-well plates in total were transferred.

Under physiological conditions, the presence of alpha-hemolysin leads to lysis of red blood cells, and the release of lysates leads to a change in the color of the supernatant. An anti-alpha-hemolysin antibody in the supernatant secreted by cells can inhibit alpha-hemolysin from lysing red blood cells. With the detection of absorbance of the supernatant, the degree of lysis of the cells caused by alpha-hemolysin and the inhibition of the antibody on alpha-hemolysin can be detected.

Hemolysis detection was performed on the culture supernatants of the 96-well plates after plate transferring, comprising following steps: the WT alpha-Toxin was diluted to obtain a stock solution with a concentration of 5 μg/mL; 25 μL of the stock solution was taken and mixed with an equal volume of cell culture supernatant, and the mixture was added into a 96-well plate containing 5% rabbit erythrocytes per well (diluted to 75 μL in PBS); and the 96-well plate was placed in an incubator at 37° C. for incubation for 1 h. Subsequently, the 96-well plate was centrifuged in a centrifuge at 3000 rpm for 3 min. 75 μL of the supernatant was added to a new 96-well plate and OD405 and OD450 were measured using a microplate reader. Part of the results is shown in panel 6B.

The screened hybridoma clones secreting anti-alpha-hemolysin antibodies were added into a 96-well plate which was plated with feeder cells by Limiting Dilution. Monoclonal cells were observed and marked under a microscope after 2-3 days, and monoclonal hybridoma cells capable of secreting anti-alpha-hemolysin monoclonal antibodies were screened by an ELISA assay after 7 days.

The monoclonal hybridoma cells secreting anti-alpha-hemolysin monoclonal antibodies were subjected to expanded culture, and total RNA of the cells was extracted using RNAfast200 Kit (Shanghai Flytech Biotechnology Co., Ltd.) according to the steps described in Kit instructions; the total RNA of the hybridoma cells obtained was reverse transcribed to cDNA using 5×primeScript RT Master Mix (Takara); and sequences of antibody light chain variable region, IgVL (κ) and heavy chain variable region V_{H} were amplified using degenerate primers (Anke Krebber., 199) and Extaq PCR reagents (Takara). PCR amplification products were purified using PCR Clean-up Gel Extraction Kit (Macherey-Nagel GmbH & Co.); and linked to T-vector using pClone007 Simple Vector Kit (Tsingke Biotechnology Co., Ltd.) according to Kit instructions, and transformed into competent Escherichia coli cells. Variable region sequences of the monoclonal antibodies were obtained by DNA sequencing after strain amplification and plasmid extraction.

Variable region sequences of murine antibodies obtained were analyzed as follows:

> murine antibody 98G9
Heavy chain variable region:
(SEQ ID NO: 44)
DVQLVESGGGLVQPGGSRKLSCAASGFTFSTFGMHWVRQAPEKGLEW
VAYISGGSSTIYYADTVKGRFTISRDNPKNTLFLQMTSLRSEDTAMY
YCASGYPYGLDYWGQGTSVTVSS
Light chain variable region:
(SEQ ID NO: 46)
DIDMTQSPSSMYASLGERVTITCKASQDINWYLSWFQQKPGKSPKTL
IYRGNRLVDGVPSRFSGSGSGQDYSLTISSLEYEDMGIYYCLQYDEF
PFTFGSGTKLEIK

Heavy and light chain CDRs were defined for murine antibody 98G9 using different definition tools, and are shown in Table 2.

TABLE 2
CDR sequences of murine antibody 98G9

| Definition | Heavy chain CDR1 | Heavy chain CDR2 | Heavy chain CDR3 |
|---|---|---|---|
| Chothia | SEQ ID NO: 1 | SEQ ID NO: 4 | SEQ ID NO: 7 |
| AbM | SEQ ID NO: 2 | SEQ ID NO: 5 | SEQ ID NO: 7 |
| Kabat | SEQ ID NO: 3 | SEQ ID NO: 6 | SEQ ID NO: 7 |
| Contact | SEQ ID NO: 2 | SEQ ID NO: 6 | SEQ ID NO: 7 |
| Definition | Light chain CDR1 | Light chain CDR2 | Light chain CDR3 |
| Chothia | SEQ ID NO: 8 | SEQ ID NO: 9 | SEQ ID NO: 10 |
| AbM | SEQ ID NO: 8 | SEQ ID NO: 9 | SEQ ID NO: 10 |
| Kabat | SEQ ID NO: 8 | SEQ ID NO: 9 | SEQ ID NO: 10 |
| Contact | SEQ ID NO: 8 | SEQ ID NO: 9 | SEQ ID NO: 10 |

> murine antibody 78F4
Heavy chain variable region:
(SEQ ID NO: 52)
QVQLQQPGAELVRPGASVKLSCKASGYSFTSYWMNWVKQRPGQGLEW
IGMIHPSDSETRLSQKFKDKATLTVDKSSSTAYMQLSSPTSEDSAVY
YCTRFDWDRAMDYWGQGTSVTVSS
Light chain variable region:
(SEQ ID NO: 54)
DIQMTQSPASLSASVGETVTITCRASENIFSYLAWYQQKQGKSPQLL
VYNTRSLAEGVPSRFSGSGSGTQFSLKINSLQPEDFGTYYCQHHYGT
PWTFGGGTKLEIK

Heavy and light chain CDRs were defined for murine antibody 78F4 using different definition tools, and are shown in Table 3.

TABLE 3
CDR sequences of murine antibody 78F4

| Definition | Heavy chain CDR1 | Heavy chain CDR2 | Heavy chain CDR3 |
|---|---|---|---|
| Chothia | SEQ ID NO: 12 | SEQ ID NO: 15 | SEQ ID NO: 18 |
| AbM | SEQ ID NO: 13 | SEQ ID NO: 16 | SEQ ID NO: 18 |
| Kabat | SEQ ID NO: 14 | SEQ ID NO: 17 | SEQ ID NO: 18 |
| Contact | SEQ ID NO: 13 | SEQ ID NO: 17 | SEQ ID NO: 18 |
| Definition | Light chain CDR1 | Light chain CDR2 | Light chain CDR3 |
| Chothia | SEQ ID NO: 19 | SEQ ID NO: 20 | SEQ ID NO: 21 |
| AbM | SEQ ID NO: 19 | SEQ ID NO: 20 | SEQ ID NO: 21 |
| Kabat | SEQ ID NO: 19 | SEQ ID NO: 20 | SEQ ID NO: 21 |
| Contact | SEQ ID NO: 19 | SEQ ID NO: 20 | SEQ ID NO: 21 |

> murine antibody 78D4
Heavy chain variable region:
(SEQ ID NO: 60)
EVHLQQSGPELMKPGASVKISCKTSGYTFSEYTMHWVKQSHGKSLEW
IGSINPNNGGTTYNQKFKGKATLTVDKSSSTAYMELRSLTSEDSAVY
NCARTRDYDNDGGLFAYWGQGTLVTVSA
Light chain variable region:
(SEQ ID NO: 62)
DVQITQSPSYLAASPGETITINCRASKNISKYLAWYQEKPGKTNKLL
IYSGSTLQSGIPSRFSGNRSGTDFTLTISSLEPEDFAMYYCQQHYEY
PFTFGGGTKLEIK

Heavy and light chain CDRs were defined for murine antibody 78D4 using different definition tools, and are shown in Table 4.

TABLE 4
CDR sequences of murine antibody 78D4

| Definition | Heavy chain CDR1 | Heavy chain CDR2 | Heavy chain CDR3 |
|---|---|---|---|
| Chothia | SEQ ID NO: 22 | SEQ ID NO: 25 | SEQ ID NO: 29 |
| AbM | SEQ ID NO: 23 | SEQ ID NO: 26 | SEQ ID NO: 29 |
| Kabat | SEQ ID NO: 24 | SEQ ID NO: 27 | SEQ ID NO: 29 |
| Contact | SEQ ID NO: 23 | SEQ ID NO: 27 | SEQ ID NO: 29 |
| Definition | Light chain CDR1 | Light chain CDR2 | Light chain CDR3 |
| Chothia | SEQ ID NO: 30 | SEQ ID NO: 32 | SEQ ID NO: 33 |
| AbM | SEQ ID NO: 30 | SEQ ID NO: 32 | SEQ ID NO: 33 |
| Kabat | SEQ ID NO: 30 | SEQ ID NO: 32 | SEQ ID NO: 33 |
| Contact | SEQ ID NO: 30 | SEQ ID NO: 32 | SEQ ID NO: 33 |

> murine antibody 16H4
Heavy chain variable region:
(SEQ ID NO: 68)
EVQLQQSGAELVKPGASVTLSCTVSGFNIKDTYMHWVKQRPEQGLEW
IGKIDPASGNTKYDPQFQGKATITADTSSNTAYLHLSSLTSEDSAVY
FCASPYGNDYAMNYWGQGTSVTVSS
Light chain variable region:
(SEQ ID NO: 70)
DIQMTQSPASLSASVGETVTITCRASEKIYSFLAWYQQKQEKSPQLL
VYNAETLAEGVPSRFSGTGSGIQFSLKIISLQPEDFGIYYCQHHYGT
PYTFGGGTKLEIK

Heavy and light chain CDRs were defined for murine antibody 16H4 using different definition tools, and are shown in Table 5.

TABLE 5
CDR sequences of murine antibody 16H4

| Definition | Heavy chain CDR1 | Heavy chain CDR2 | Heavy chain CDR3 |
|---|---|---|---|
| Chothia | SEQ ID NO: 34 | SEQ ID NO: 37 | SEQ ID NO: 40 |
| AbM | SEQ ID NO: 35 | SEQ ID NO: 38 | SEQ ID NO: 40 |
| Kabat | SEQ ID NO: 36 | SEQ ID NO: 39 | SEQ ID NO: 40 |
| Contact | SEQ ID NO: 35 | SEQ ID NO: 39 | SEQ ID NO: 40 |
| Definition | Light chain CDR1 | Light chain CDR2 | Light chain CDR3 |
| Chothia | SEQ ID NO: 41 | SEQ ID NO: 42 | SEQ ID NO: 43 |
| AbM | SEQ ID NO: 41 | SEQ ID NO: 42 | SEQ ID NO: 43 |
| Kabat | SEQ ID NO: 41 | SEQ ID NO: 42 | SEQ ID NO: 43 |
| Contact | SEQ ID NO: 41 | SEQ ID NO: 42 | SEQ ID NO: 43 |

### EXAMPLE 8

### Binding Affinity of Antibodies of the Present Invention to Alpha-Hemolysin (Alpha-Toxin)

Alpha-hemolysin was diluted to 1 μg/mL with PBS, then 100 μL per well was added into a 96-well plate (Microwell 96F 167008, Thermo) and incubated at 4° C. overnight for coating. Next day, the 96-well plate was washed with PBST (containing 0.5% PBS), for which the wells were soaked with PBST for 1 min each time, and then residual water was thoroughly spun off. 200 μL of PBST containing 5% BSA was added into the wells of the plate respectively, which then was incubated at 37° C. for 1 h for blocking; subsequently, the plate was washed with PBST and then residual water in the wells was thoroughly spun off.

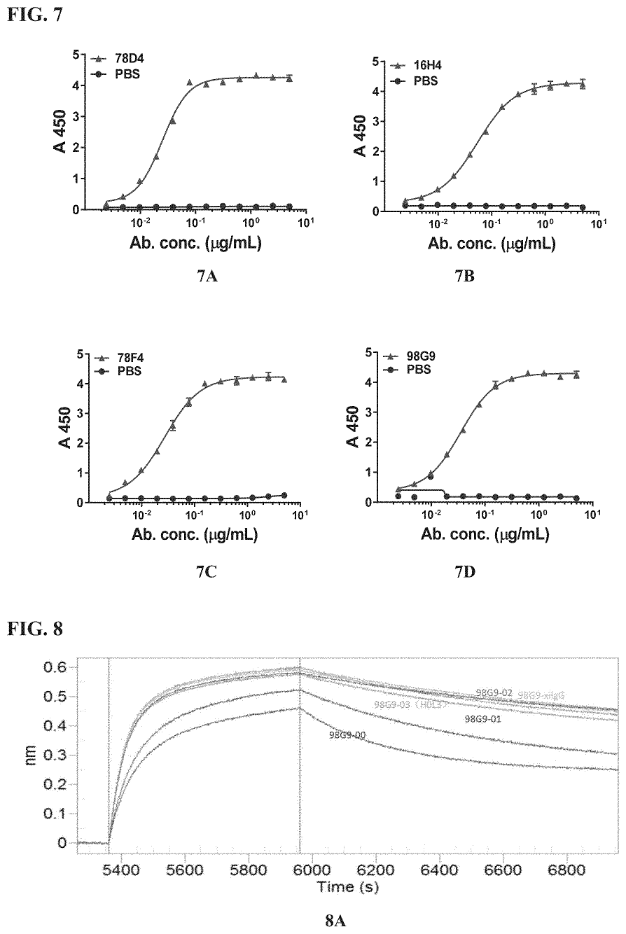

100 μL of each of recombinantly expressed anti-alpha-hemolysin monoclonal antibodies of different concentrations obtained through serial dilution (see the abscissae in FIG. 7 for antibody concentrations) was added to the 96-well plate per well, which then was incubated at 4° C. overnight. Then the 96-well plate was washed with PBST, before 100 μL of anti-mouse IgG secondary antibody (IH-0031, Beijing Dingguo Changsheng Biotechnology CO. LTD.) was added into each well, and the plate was incubated for 1 h at 37° C. Washed with PBST for 5 times, the plate was added with 100 μL of Substrate Solution (Invitrogen) per well, and incubated at 37° C. for 10 min. The reaction was stopped by the addition of 50 μL of 2 N sulfuric acid per well, and absorbance at a wavelength of 450 nm was measured in a microplate reader (Multiskein FC, Thermo).

The results are shown in FIG. 7.

### EXAMPLE 9

### Obtainment of Chimeric Antibodies and Humanized Antibodies of the Present Invention

First, complete light and heavy chain variable regions of the murine antibodies were combined with the human light and heavy chain constant regions to obtain chimeric antibodies as controls. Each obtained chimeric antibody was named following a format “murine antibody abbreviation-xi”.

With a comprehensive analysis of the heavy chain sequence of each murine antibody, Complementarity Determining Regions (CDRs) of the antibody binding to the antigen as well as Framework Regions (FRs) supporting the conserved three-dimensional conformation of the antibody were determined. Sequences of known human antibodies were searched and analyzed, and a heavy chain sequence of human antibody which was most similar and closest to the murine antibody was selected, such as IGHV1-3*01, and the sequences of framework regions in it were selected as templates. The heavy chain CDRs of the murine antibody were grafted into the framework regions of the human antibody to generate a humanized antibody heavy chain sequence (heavy chain version 0). Subsequently, residues at individual amino acid positions corresponding to positions in the murine framework regions that may be involved in antigen-antibody binding were back mutated to original murine residues, to generate humanized antibody heavy chain sequences (versions 1, 2, 3, . . . ). Humanized antibody light chain sequences (version 0, 1, 2, . . . ) were generated by applying the same procedure. Humanized antibody light and heavy chain sequences were designed, synthesized, and co-transfected into HEK 293 cells and humanized antibodies were expressed recombinantly (versions were named as follows: e.g., H0L0 was obtained by co-expressing heavy chain version 0+light chain version 0, and could be further abbreviated as version 00). Experiments proved that purified humanized antibodies showed activities of specifically binding to alpha-hemolysin protein consistent to those of murine parent antibodies respectively.

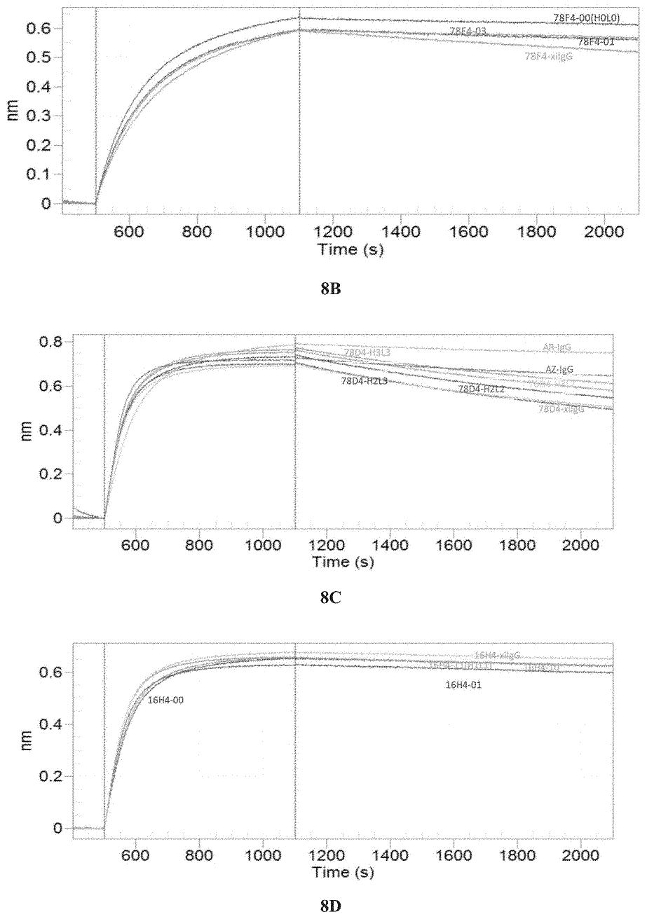

Those finally obtained humanized antibody versions and chimeric antibody xi versions were compared for antigen binding using an Octet instrument, and FIG. 8 shows measurement results of certain antibodies. From the curves of association and dissociation phases of binding of the antibodies to the antigen, certain humanized antibody versions shows similar or better properties in the antibody-antigen association and dissociation phases than the control antibodies ('including chimeric antibodies of the present invention or antibodies AZ, and AR).

Humanized antibodies having following sequences were obtained by screening.

> humanized antibody 98G9-02(98G9-H0L2)
Heavy chain variable region (H0):
(SEQ ID NO: 48)
EVQLVESGGGLVQPGGSLRLSCAASGFTFSTFGMHWVRQAPGKGLEW
VSYISGGSSTIYYADTVKGRFTISRDNAKNSLYLQMNSLRAEDTAVY
YCASGYPYGLDYWGQGTLVTVSS
Light chain variable region (L2):
(SEQ ID NO: 76)
DIQMTQSPSSLSASVGDRVTITCKASQDINWYLSWFQQKPGKAPKTL
IYRGNRLVDGVPSRFSGSGSGQDYTFTISSLQPEDMATYYCLQYDEF
PFTFGQGTKVEIK
> humanized antibody98G9-03 (98G9-H0L3)
Heavy chain variable region (H0):
(SEQ ID NO: 48)
EVQLVESGGGLVQPGGSLRLSCAASGFTFSTFGMHWVRQAPGKGLEW
VSYISGGSSTIYYADTVKGRFTISRDNAKNSLYLQMNSLRAEDTAVY
YCASGYPYGLDYWGQGTLVTVSS
Light chain variable region (L3):
(SEQ ID NO: 50)
DIQMTQSPSSLSASVGDRVTITCKASQDINWYLSWFQQKPGKAPKTL
IYRGNRLVEGVPSRFSGSGSGQDYTFTISSLQPEDMATYYCLQYDEF
PFTFGQGTKVEIK
> humanized antibody78F4-00 (78F4-H0L0)
Heavy chain variable region (H0):
(SEQ ID NO: 56)
QVQLVQSGAEVKKPGASVKVSCKASGYSFTSYWMNWVRQAPGQGLEW
MGMIHPSDSETRLSQKFKDRVTMTRDTSTSTVYMELSSLRSEDTAVY
YCARFDWDRAMDYWGQGTLVTVSS
Light chain variable region (L0):
(SEQ ID NO: 58)
DIQMTQSPSSLSASVGDRVTITCRASENIFSYLAWYQQKPGKAPKLL
IYNTRSLAEGVPSRFSGSGSGTDFTLTISSLQPEDFATYYCQHHYGT
PWTFGQGTKLEIK
> humanized antibody78D4-33 (78D4-H3L3)
Heavy chain variable region (H3):
(SEQ ID NO: 64)
QVQLVQSGAEVKKPGASVKVSCKTSGYTFSEYTMHWVRQAPGQRLEW
MGSINPNQGGTTYNQKFKGRVTITVDKSASTAYMELSSLRSEDTAVY
YCARTRDYDNDGGLFAYWGQGTLVTVSS
Light chain variable region (L3):
(SEQ ID NO: 66)
DVQITQSPSFLSASVGDRVTITCRASKNILKYLAWYQQKPGKAPKLL
IYSGSTLQSGVPSRFSGSRSGTEFTLTISSLQPEDFATYYCQQHYEY
PFTFGQGTKLEIK
> humanized antibody16H14-11 (16H4-H1L1)
Heavy chain variable region (H1):
(SEQ ID NO: 72)
EVQLVQSGAEVKKPGATVKISCKVSGFNIKDTYMHWVQQAPGKGLEW
MGKIDPASGNTKYDPQFQGRVTITADTSTNTAYMELSSLRSEDTAVY
YCATPYGNDYAMNYWGQGTLVTVSS
Light chain variable region (L1):
(SEQ ID NO: 74)
DIQMTQSPSSLSASVGDRVTITCRASEKIYSFLAWYQQKPGKAPKLL
LYNAETLAEGVPSRFSGSGSGIDFTLTISSLQPEDFATYYCQHHYGT
PYTFGGGTKVEIK

Heavy and light chain CDRs of the humanized antibodies are shown in Table 6.

TABLE 6
CDR sequences of humanized antibodies

| 98G9 H0L2 | heavy chain CDR1 | heavy chain CDR2 | heavy chain CDR3 |
|---|---|---|---|
| | SEQ ID NO: 2 | SEQ ID NO: 6 | SEQ ID NO: 7 |
| | light chain CDR1 | light chain CDR2 | light chain CDR3 |
| | SEQ ID NO: 8 | SEQ ID NO: 9 | SEQ ID NO: 10 |
| 98G9 H0L3 | heavy chain CDR1 | heavy chain CDR2 | heavy chain CDR3 |
| | SEQ ID NO: 2 | SEQ ID NO: 6 | SEQ ID NO: 7 |
| | light chain CDR1 | light chain CDR2 | light chain CDR3 |
| | SEQ ID NO: 8 | SEQ ID NO: 11 | SEQ ID NO: 10 |
| 78F4 H0L0 | heavy chain CDR1 | heavy chain CDR2 | heavy chain CDR3 |
| | SEQ ID NO: 13 | SEQ ID NO. 17 | SEQ ID NO: 18 |
| | light chain CDR1 | light chain CDR2 | light chain CDR3 |
| | SEQ ID NO: 19 | SEQ ID NO: 20 | SEQ ID NO: 21 |
| 78D4 H3L3 | heavy chain CDR1 | heavy chain CDR2 | heavy chain CDR3 |
| | SEQ ID NO: 23 | SEQ ID NO: 28 | SEQ ID NO: 29 |
| | light chain CDR1 | light chain CDR2 | light chain CDR3 |
| | SEQ ID NO: 31 | SEQ ID NO: 32 | SEQ ID NO: 33 |
| 16H4 H1L1 | heavy chain CDR1 | heavy chain CDR2 | heavy chain CDR3 |
| | SEQ ID NO: 35 | SEQ ID NO: 39 | SEQ ID NO: 40 |
| | light chain CDR1 | light chain CDR2 | light chain CDR3 |
| | SEQ ID NO: 41 | SEQ ID NO: 42 | SEQ ID NO: 43 |

### EXAMPLE 10

### Detection of Antihemolytic Activity of Antibodies of the Present Invention Against Alpha-Hemolysin (Alpha-Toxin)

75 μL of 5% rabbit erythrocytes (Bio-channel Biotechnology) and 12.5 ng Staphylococcus aureus alpha-hemolysin and different amounts of anti-alpha-hemolysin antibodies (12.5 ng, 25 ng, and 50 ng) were added to the wells of a 96-well plate, and PBS was added to obtain a final volume of 150 μL. The 96-well plate was incubated in an incubator at 37° C. for 1 h, and then was centrifuged at 3000 rpm for 3 min. 100 μL of the supernatant from each well was evaluated for hemolytic activity by absorbance at 405 nm (OD405) measured using a microplate reader.

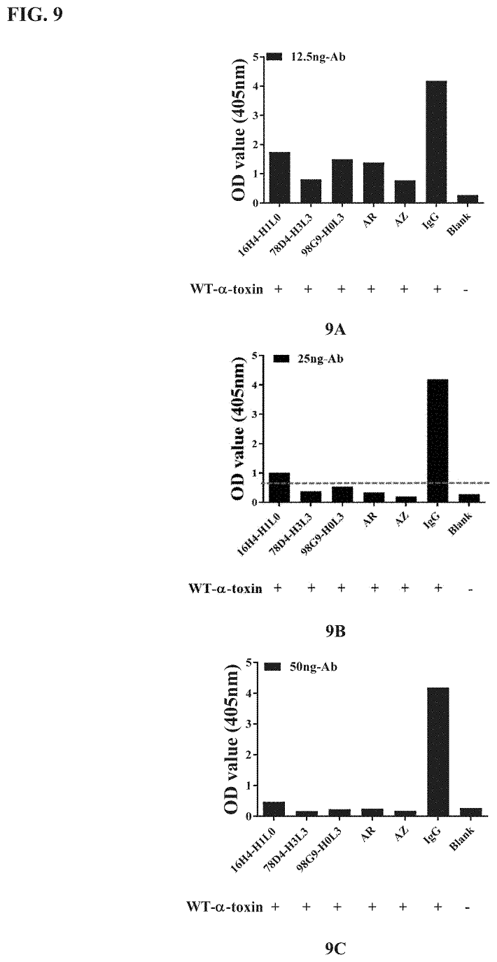

The experimental results showed that the chimeric antibodies and humanized antibodies of the present invention had obvious antihemolytic activities against alpha-hemolysin, which were dose dependent, and certain antibodies had comparable activities to control antibodies AR and AZ. The results are shown in FIG. 9.

### EXAMPLE 11

### Detection of Binding Kinetics (Kₒₙ, K_{off}) and Affinity Constant KD of Antibodies of the Present Invention to Alpha-Hemolysin (Alpha-Toxin)

Antibody-antigen interactions were measured using BIAcore S200 from GE Healthcare.

Referring to the instructions provided in Human Antibody Capture Kit (cat No. BR-1008-39, Lot 10261753) from GE Healthcare, the analytical channel and the control sample channel on CM5 sensor chip were first saturated and coupled with maximum amount of anti-human Fc antibody, then the antibody to be detected at 7.5 ug/ml was allowed to flow through the analytical channel and uniformly distribute; and finally antigen samples gradiently diluted (the initial concentration was 20 nM and diluted by 1:3 to get 8 concentrations, and the concentration 0.741 nM was set to be repeated) were allowed to flow through both the analytical channel and the sample channel, and the photoreactions upon the binding of antibody to antigen were measured. Subsequently, the association constant Kon and dissociation constant Koff and affinity constant KD of the antibody were finally obtained by instrument software fitting (1:1) analysis.

The results are shown in Table 7.

TABLE 7
Binding kinetics and affinity constants of antibodies of the present invention to antigen

| Antibody | ka (1/Ms) | kd (1/s) | KD (M) | Rmax (RU) | Chi² (RU²) |
|---|---|---|---|---|---|
| 78D4 xilgG | 1 40E+06 | 3.36E−04 | 2.39E−10 | 67.1 | 0.198 |
| 78D4-H3L3 | 1.25E+06 | 3.02E−04 | 2.40E−10 | 37.2 | 0.0303 |
| 98G9 xilgG | 8.84E+05 | 3.77E−04 | 4.26E−10 | 47.3 | 0.46 |
| 98G9-H0L2 | 7.76E+05 | 5.24E−04 | 6.74E−10 | 18.9 | 0.0677 |
| 98G9-H0L3 | 7.19E+05 | 5.12E−04 | 7.13E−10 | 22.5 | 0.0481 |
| AZ IgG | 1.60E+06 | 2.11E−04 | 1.32E−10 | 48.8 | 0.0552 |
| AR IgG | 2.76E+05 | 6.77E−05 | 2.45E−10 | 48.7 | 0.48 |

### EXAMPLE 12

### Establishment of Animal Model of Alpha-Hemolysin-Induced Sepsis and Detection of Therapeutic Effects of Antibodies of the Present Invention

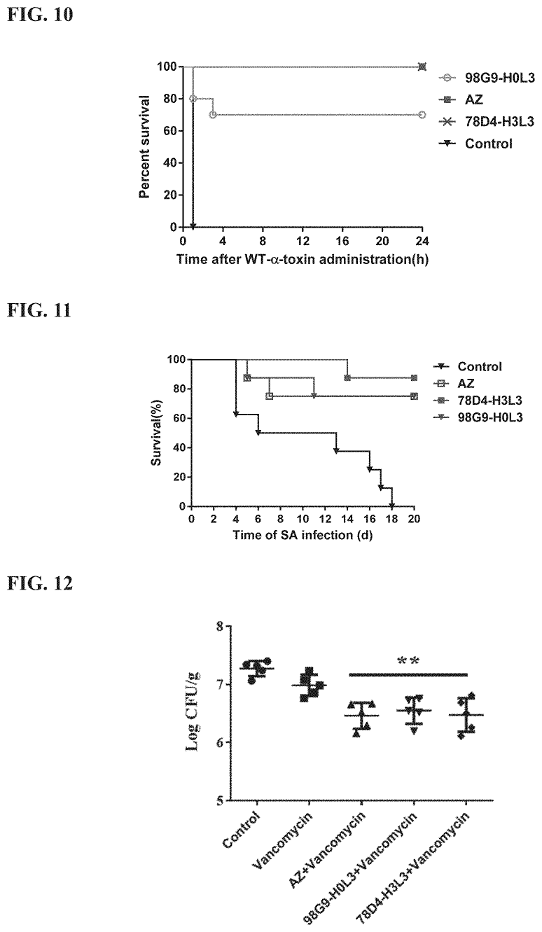

C57BL/6J mice were randomly divided into a model control group and monoclonal antibody drug treatment groups according to body weight. 30 min before experiment, mice in the treatment groups were injected with a corresponding anti-alpha-hemolysin monoclonal antibody (6 μg/mouse) via tail vein respectively, and mice in the control group were injected with the same dose of PBS; and then all the mice were injected with alpha-hemolysin (3 μg/mouse) via tail vein to establish a sepsis infected mouse model. The survival time of the experimental animals was observed and recorded, and the results are shown in FIG. 10.

### EXAMPLE 13

### Establishment of Animal Model of Methicillin-Resistant Staphylococcus aureus-Induced Bacteremia and Detection of Therapeutic Effects of Antibodies of the Present Invention

A methicillin-resistant Staphylococcus aureus USA300 strain was activated for 2 generations on a solid TSB plate, and then inoculated into a liquid TSB medium for overnight culture. The bacterial cells were collected by being centrifuged at 12,000 rpm, and resuspended in physiological saline for later use

C257BL/6J mice were infected with 6×10⁷ CFU/mouse USA300 via tail vein and were randomly divided into a model control group (Control) and anti-alpha-hemolysin monoclonal antibody drug treatment groups according to body weight. 2 h after infection, the mice in the monoclonal antibody drug treatment groups were administered by tail vein injection a dosage of 5 mg/kg of a corresponding antibody respectively, and the mice in the control group were injected the same dose of PBS. The survival time of the mice in the groups was observed and recorded, and the results are shown in FIG. 11.

### EXAMPLE 14

### Establishment of Animal Model of Methicillin-Resistant Staphylococcus aureus Pneumonia and Detection of Therapeutic Effects of Antibodies of the Present Invention

A methicillin-resistant Staphylococcus aureus USA300 strain was activated for 2 generations on a solid TSB plate, and then inoculated into a liquid TSB medium for overnight culture. The bacterial cells were collected by being centrifuged at 12,000 rpm, and resuspended in physiological saline for later use.

C57BL/6J mice were infected with 1.8×10⁸ CFU/mouse USA300 via trachea and were randomly divided into a model control group, a monoclonal antibody drug treatment group, a vancomycin treatment group, and vancomycin plus monoclonal antibody treatment groups according to body weight. 2 h after infection, the mice in the groups were administered by tail vein injection corresponding drug for treatment (the dosage of the corresponding monoclonal antibody was 15 mg/kg, and the dosage of vancomycin was 1.25 mg/kg), or the same volume of PBS. The animals were killed 24 h after infection, and lung tissues were taken, weighted and homogenized, and coated on a solid TSB medium to detect bacterial load in the tissues. The experimental results showed that the anti-alpha-hemolysin antibodies of the present invention were capable of enhancing the pharmacodynamic effect of vancomycin in the treatment of methicillin-resistant Staphylococcus aureus pneumonia.

The results are shown in FIG. 12

### EXAMPLE 15

### Acute Toxicity Study of Antibodies of the Present Invention

Acute toxicity study on antibody molecule 78D4 H3L3 of the present invention was performed in mice and cynomolgus monkeys (N=10 for the experiment in mice, and N=3 for the experiment in cynomolgus monkeys).

C57BL/6 mice (18-20 g), half male and half female, were injected with 125 mg/kg of antibody molecule 78D4 H3L3 per mouse via tail vein within 24 hours. The results showed that when a dosage of 125 mg/kg at the maximum was administered in 24 hours, no mice died, and no discomfort was shown by the animals during continuous observation for 14 days, and no abnormality was seen in main organs (heart, liver, spleen, lung, kidney and brain) taken for histology observation from sacrificed animals.

Male cynomolgus monkeys were administered 10 mg/kg of antibody molecule 78D4 H3L3 per monkey via intravenous drip in limbs, in a single dose. The results showed that when a dosage of 10 mg/kg of antibody molecule 78D4 H3L3 was administrated, no death of the animals occurred, and no discomfort was shown by the animals during continuous observation for 28 days.

Acute toxicity test research showed that antibody molecule 78D4 H3L3 had good safety.

### EXAMPLE 16

### Pharmacokinetic Study of Antibodies of the Present Invention

Antibody molecule 78D4 H3L3, at a dose of 10 mg/kg, was subjected to a single dose pharmacokinetic study in cynomolgus monkeys (N=3).

Male cynomolgus monkeys were administered 10 mg/kg of antibody molecule 78D4 H3L3 per monkey via intravenous drip in limbs, in a single dose. Blood was sampled at 0 h before administration (pre-dose, 0 h), and at 0.25 h (15 min), 0.5 h (time point when needle was withdrawn), 4 h, 24 h (D2), 48 h (D3), 96 h (D5), 168 h (D8), 336 h (D15), 504 h (D22), and 672 h (D29) after administration started. Blood sampling sites were in peripheral veins in limbs of the animals (not the administered limb), or inguinal veins of the animals. The blood samples were about 1 mL whole blood/animal/time point. Antibody concentrations in the sera from the cynomolgus monkeys were determined using ELISA, and pharmacokinetic parameters such as AUClast, CL, T1/2 and the like were calculated by Phoenix WinNonlin (v6.4, Pharsight, Inc.) software using non-compartmental analysis methods.

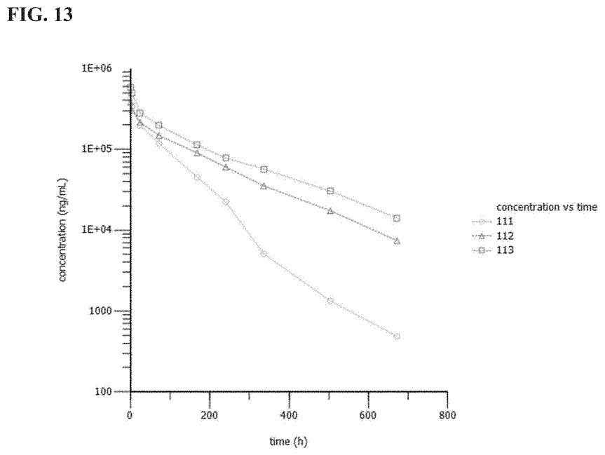

The concentrations of antibody 78D4 H3L3 in sera from the cynomolgus monkeys were determined using ELISA. Serum drug levels of individual animals are shown in FIG. 13, and pharmacokinetic parameters are summarized in Table 8. Drug exposure can be seen with each animal after administration.

TABLE 8
Pharmacokinetic results of antibody 78D4 H3L3

| | | Estimate | | | | | | |
|---|---|---|---|---|---|---|---|---|
| | | Animal ID | | | | | | |
| Parameter | Unit | 111 | 112 | 113 | N | Mean | SD | CV % |
| AUC_{INF}_obs | h*ng/mL | 26900000 | 44700000 | 64300000 | 3 | 45300000 | 18700000 | 41.3 |
| AUCₗₐₛₜ | h*ng/mL | 26800000 | 43200000 | 60700000 | 3 | 43600000 | 17000000 | 38.9 |
| C₀ | ng/mL | 431000 | 387000 | 592000 | 3 | 470000 | 108000 | 22.9 |
| Cl__{obs} | mL/h/kg | 0.744 | 0.447 | 0.311 | 3 | 0.501 | 0.222 | 44.2 |
| MRTₗₐₛₜ | h | 83.8 | 160 | 170 | 3 | 138 | 47.1 | 34.2 |
| T_{1/2z} | h | 99.2 | 140 | 173 | 3 | 137 | 36.9 | 26.9 |
| V_{z} _obs | mL/kg | 107 | 90.4 | 77.6 | 3 | 91.5 | 14.5 | 15.8 |

The above description of the embodiments of the present invention is not intended to limit the present invention, and those skilled in the art may make various changes and modifications to the present invention without departing from the spirit of the present invention, which should fall within the scope of the appended claims.

1. A method for preparing an anti-alpha-hemolysin monoclonal antibody, including steps as follows:
(1) immunizing animals with an attenuated alpha-hemolysin or an alpha-hemolysin without virulence;
(2) taking splenocytes and preparing hybridomas;
(3) screening an antibody having high affinity to virulent alpha-hemolysin or wild-type alpha-hemolysin using the same;
(4) selecting an antibody which has an activity of inhibiting erythrocyte lysis caused by virulent alpha-hemolysin or wild-type alpha-hemolysin through an assay of inhibition of erythrocyte lysis.

2. The method for preparing an anti-alpha-hemolysin monoclonal antibody according to claim 1, wherein the virulent alpha-hemolysin or wild-type alpha-hemolysin is Staphylococcus aureus alpha-hemolysin, and preferably has an amino acid sequence as shown in SEQ ID NO: 78; and the attenuated alpha-hemolysin or alpha-hemolysin without virulence is alpha-hemolysin with H35L, and preferably has an amino acid sequence as shown in SEQ ID NO: 80.

3. The method for preparing an anti-alpha-hemolysin monoclonal antibody according to claim 1, wherein the virulent alpha-hemolysin or wild-type alpha-hemolysin, the attenuated alpha-hemolysin, or the alpha-hemolysin without virulence is independently linked to a purification tag, a carrier protein, and/or an adjuvant molecule;
and the linkage includes coupling, cross-linking, conjugation, and/or fusion.

4. An anti-alpha-hemolysin monoclonal antibody prepared by the method according to claim 1, wherein the antibody has at least one activity selected from the group consisting of blocking hemolytic effect of alpha-hemolysin, blocking damage of alpha-hemolysin to lung epithelial cells, reducing bacterial load in MRSA infected lung tissue, prolonging survival time of a patient suffering from MRSA bacteremia, and preventing or alleviating sepsis caused by alpha-hemolysin.

5. An anti-alpha-hemolysin monoclonal antibody prepared by the method according to any one of claims 1 to 3 claim 1, wherein the antibody has
VH-CDR1: selected from the group consisting of SEQ ID NOs: 1, 2, 3, 12, 13, 14, 22, 23, 24, 34, 35, and 36;
VH-CDR2: selected from the group consisting of SEQ ID NOs: 4, 5, 6, 15, 16, 17, 25, 26, 27, 28, 37, 38, and 39;
VH-CDR3: selected from the group consisting of SEQ ID NOs: 7, 18, 29, and 40;
VL-CDR1: selected from the group consisting of SEQ ID NOs: 8, 19, 30, 31, and 41;
VL-CDR2: selected from the group consisting of SEQ ID NOs: 9, 11, 20, 32, and 42; and
VL-CDR3: selected from the group consisting of SEQ ID NOs: 10, 21, 33, and 43.

6. An antibody or fragment thereof comprising a heavy chain variable region (VH) and a light chain variable region (VL), wherein the heavy chain variable region (VH) and the light chain variable region (VL) comprise a combination of CDRs (VH-CDR1, VH-CDR2, VH-CDR3; and VL-CDR1, VL-CDR2, VL-CDR3) selected from the group consisting of:
(1) VH-CDR1 as shown in SEQ ID NO: 1, VH-CDR2 as shown in SEQ ID NO: 4, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;
(2) VH-CDR1 as shown in SEQ ID NO: 2, VH-CDR2 as shown in SEQ ID NO: 5, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;
(3) VH-CDR1 as shown in SEQ ID NO: 3, VH-CDR2 as shown in SEQ ID NO: 6, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;
(4) VH-CDR1 as shown in SEQ ID NO: 2, VH-CDR2 as shown in SEQ ID NO: 6, and VH-CDR3 as shown in SEQ ID NO: 7; and, VL-CDR1 as shown in SEQ ID NO: 8, VL-CDR2 as shown in SEQ ID NO: 9, and VL-CDR3 as shown in SEQ ID NO: 10;
(5) VH-CDR1 as shown in SEQ ID NO: 2, VH-CDR2 as shown in SEQ ID NO: 6, and VH-CDR3 as shown in SEQ ID NO: 7; and, VL-CDR1 as shown in SEQ ID NO: 8, VL-CDR2 as shown in SEQ ID NO: 11, and VL-CDR3 as shown in SEQ ID NO: 10;
(6) VH-CDR1 as shown in SEQ ID NO: 12, VH-CDR2 as shown in SEQ ID NO: 15, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;
(7) VH-CDR1 as shown in SEQ ID NO: 13, VH-CDR2 as shown in SEQ ID NO: 16, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;
(8) VH-CDR1 as shown in SEQ ID NO: 14, VH-CDR2 as shown in SEQ ID NO: 17, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;
(9) VH-CDR1 as shown in SEQ ID NO: 13, VH-CDR2 as shown in SEQ ID NO: 17, and VH-CDR3 as shown in SEQ ID NO: 18; and, VL-CDR1 as shown in SEQ ID NO: 19, VL-CDR2 as shown in SEQ ID NO: 20, and VL-CDR3 as shown in SEQ ID NO: 21;
(10) VH-CDR1 as shown in SEQ ID NO: 22, VH-CDR2 as shown in SEQ ID NO: 25, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;
(11) VH-CDR1 as shown in SEQ ID NO: 23, VH-CDR2 as shown in SEQ ID NO: 26, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;
(12) VH-CDR1 as shown in SEQ ID NO: 24, VH-CDR2 as shown in SEQ ID NO: 27, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;
(13) VH-CDR1 as shown in SEQ ID NO: 23, VH-CDR2 as shown in SEQ ID NO: 27, and VH-CDR3 as shown in SEQ ID NO: 29; and, VL-CDR1 as shown in SEQ ID NO: 30, VL-CDR2 as shown in SEQ ID NO: 32, and VL-CDR3 as shown in SEQ ID NO: 33;
(14) VH-CDR1 as shown in SEQ ID NO: 23, VH-CDR2 as shown in SEQ ID NO: 28, and VH-CDR3 as shown in SEQ ID NO: 29; and, VL-CDR1 as shown in SEQ ID NO: 31, VL-CDR2 as shown in SEQ ID NO: 32, and VL-CDR3 as shown in SEQ ID NO: 33;
(15) VH-CDR1 as shown in SEQ ID NO: 34, VH-CDR2 as shown in SEQ ID NO: 37, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43;
(16) VH-CDR1 as shown in SEQ ID NO: 35, VH-CDR2 as shown in SEQ ID NO: 38, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43;
(17) VH-CDR1 as shown in SEQ ID NO: 36, VH-CDR2 as shown in SEQ ID NO: 39, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43; and
(18) VH-CDR1 as shown in SEQ ID NO: 35, VH-CDR2 as shown in SEQ ID NO: 39, and VH-CDR3 as shown in SEQ ID NO: 40; and, VL-CDR1 as shown in SEQ ID NO: 41, VL-CDR2 as shown in SEQ ID NO: 42, and VL-CDR3 as shown in SEQ ID NO: 43.

7. The antibody or fragment thereof according to claim 6, wherein the heavy chain variable region comprises:
an amino acid sequence as shown in any one of SEQ ID NO: 44, SEQ ID NO: 48, SEQ ID NO: 52, SEQ ID NO: 56, SEQ ID NO: 60, SEQ ID NO: 64, SEQ ID NO: 68 and SEQ ID NO: 72, or an amino acid sequence having at least 75% identity to the amino acid sequence as shown; and/or
the light chain variable region comprises:
an amino acid sequence as shown in any one of SEQ ID NO: 46, SEQ ID NO: 50, SEQ ID NO: 54, SEQ ID NO: 58, SEQ ID NO: 62, SEQ ID NO: 66, SEQ ID NO: 70, SEQ ID NO: 74 and SEQ ID NO: 76 or an amino acid sequence having at least 75% identity to the amino acid sequence as shown.

8. The antibody or fragment thereof according to claim 7, wherein the antibody or fragment thereof comprises a heavy chain variable region and a light chain variable region selected from the group consisting of following combinations:
(1) an amino acid sequence as shown in SEQ ID NO: 44 or an amino acid sequence having at least 75% identity to the amino acid sequence as shown in SEQ ID NO: 44;
and, an amino acid sequence as shown in SEQ ID NO: 46 or an amino acid sequence having at least 75% identity to the amino acid sequence as shown in SEQ ID NO: 46;
(2) an amino acid sequence as shown in SEQ ID NO: 48 or an amino acid sequence having at least 75% identity to the amino acid sequence as shown in SEQ ID NO: 48;
and, an amino acid sequence as shown in SEQ ID NO: 50 or an amino acid sequence having at least 75% identity to the amino acid sequence as shown in SEQ ID NO: 50;
(3) an amino acid sequence as shown in SEQ ID NO: 52 or an amino acid sequence having at least 75% identity to the amino acid sequence as shown in SEQ ID NO: 52;
and, an amino acid sequence as shown in SEQ ID NO: 54 or an amino acid sequence having at least 75% identity to the amino acid sequence as shown in SEQ ID NO: 54;
(4) an amino acid sequence as shown in SEQ ID NO: 56 or an amino acid sequence having at least 75% identity to the amino acid sequence as shown in SEQ ID NO: 56;
and, an amino acid sequence as shown in SEQ ID NO: 58 or an amino acid sequence having at least 75% identity to the amino acid sequence as shown in SEQ ID NO: 58;
(5) an amino acid sequence as shown in SEQ ID NO: 60 or an amino acid sequence having at least 75% identity to the amino acid sequence as shown in SEQ ID NO: 60;
and, an amino acid sequence as shown in SEQ ID NO: 62 or an amino acid sequence having at least 75% identity to the amino acid sequence as shown in SEQ ID NO: 62;
(6) an amino acid sequence as shown in SEQ ID NO: 64 or an amino acid sequence having at least 75% identity to the amino acid sequence as shown in SEQ ID NO: 64;
and, an amino acid sequence as shown in SEQ ID NO: 66 or an amino acid sequence having at least 75% identity to the amino acid sequence as shown in SEQ ID NO: 66;
(7) an amino acid sequence as shown in SEQ ID NO: 68 or an amino acid sequence having at least 75% identity to the amino acid sequence as shown in SEQ ID NO: 68;
and, an amino acid sequence as shown in SEQ ID NO: 70 or an amino acid sequence having at least 75% identity to the amino acid sequence as shown in SEQ ID NO: 70;
(8) an amino acid sequence as shown in SEQ ID NO: 72 or an amino acid sequence having at least 75% identity to the amino acid sequence as shown in SEQ ID NO: 72;
and, an amino acid sequence as shown in SEQ ID NO: 74 or an amino acid sequence having at least 75% identity to the amino acid sequence as shown in SEQ ID NO: 74; and
(9) an amino acid sequence as shown in SEQ ID NO: 48 or an amino acid sequence having at least 75% identity to the amino acid sequence as shown in SEQ ID NO: 48;
and, an amino acid sequence as shown in SEQ ID NO: 76 or an amino acid sequence having at least 75% identity to the amino acid sequence as shown in SEQ ID NO: 76.

9. The antibody or fragment thereof according to claim 8, wherein the at least 75% identity is any percent identity greater than or equal to 75%, such as at least 80%, preferably at least 85%, more preferably at least 90%, even more preferably at least 91° A, 92%, 93%, 94%, 95%, 96%, 97%, 98%, or even 99% identity;
preferably, the antibody or fragment thereof is in any form, for example, a monoclonal antibody, a single-chain antibody, a single-domain antibody, a bifunctional antibody, a nanobody, a fully or partially humanized antibody or a chimeric antibody and the like;
alternatively, the antibody or fragment thereof is a half-antibody or an antigen-binding fragment of a half-antibody, for example, scFv, BsFv, dsFv, (dsFv)₂, Fab, Fab′, F(ab′)₂, or Fv;
alternatively, the antibody is IgA, IgD, IgE, IgG or IgM, more preferably IgG1; or, the fragment of the antibody is selected from the group consisting of scFv, Fab, F(ab′)₂ and Fv fragments of the antibody.

10. The antibody or fragment thereof according to claim 9, wherein the antibody or fragment thereof further comprises a human or murine light chain constant region (CL) and/or heavy chain constant region (CH); preferably, the antibody or fragment thereof comprises a heavy chain constant region selected from the group consisting of IgG, IgA, IgM, IgD and IgE and/or a kappa or lambda type light chain constant region; more preferably, the heavy chain constant region is of IgG1 or IgG4 subtype and the light chain constant region is of kappa type.

11. A nucleic acid molecule comprising a nucleotide sequence encoding the antibody or fragment thereof according to claim 10, or encoding a heavy chain CDR, a light chain CDR, a heavy chain variable region, a light chain variable region, a heavy chain or a light chain comprised in the antibody or fragment thereof.

12. A vector comprising the nucleic acid molecule according to claim 11.

13. A host cell comprising the nucleic acid molecule and/or the vector according to claim 12, or transformed or transfected with the nucleic acid molecule and/or the vector.

14. A conjugate or fusion protein comprising the antibody or fragment thereof according to claim 6.

15. The conjugate or fusion protein according to claim 14, wherein the conjugate or fusion protein comprises an additional moiety, for example, a cell surface receptor, a small molecule compound, a small molecule polymer, an active protein or a polypeptide, directly or indirectly linked to the antibody or fragment thereof.

16. A pharmaceutical composition comprising the antibody or fragment thereof according to claim 6, and optionally a pharmaceutically acceptable excipient.

17. A kit comprising the antibody or fragment thereof according to claim 6.

18. Use of the antibody or fragment thereof according to claim 4, in the manufacture of a medicament for preventing or treating infection and complication caused by alpha-hemolysin or an alpha-hemolysin producing microorganism.

19. Use of the antibody or fragment thereof according to claim 4, in combination with another antibacterial agent or anti-alpha-hemolysin antibody in the manufacture of a medicament for preventing or treating infection and complication caused by alpha-hemolysin or an alpha-hemolysin producing microorganism.

20. Use according to claim 18, wherein the microorganism is Staphylococcus aureus; preferably, the Staphylococcus aureus includes methicillin-resistant Staphylococcus aureus;
preferably, the infection includes upper respiratory tract infection, pneumonia, severe pneumonia, abdominal infection, subcutaneous and soft tissue infection, bacteremia, and infection in various organs;
preferably, the complication includes Acute Respiratory Distress Syndrome (ARDS), sepsis, and a rise in inflammatory factors in body;
preferably, the another antibacterial agent is an agent (including chemical agents, biological agents and Chinese medicines) useful for the treatment and prevention of infection caused by Staphylococcus aureus, e.g. methicillin-resistant Staphylococcus aureus, preferably is an antibiotic, such as beta-lactam antibiotics, more preferably vancomycin, norvancomycin, teicoplanin, linezolid, daptomycin, cephapiprazole, fusidic acid, or ceftaroline.

21. A method for preventing or treating infection and complication caused by alpha-hemolysin or an alpha-hemolysin producing microorganism, including administering to a subject in need thereof the antibody or fragment thereof according to claim 4, and optionally an antibacterial agent.

22. A method for diagnosing infection caused by alpha-hemolysin or an alpha-hemolysin producing microorganism, including contacting the antibody or fragment thereof according to claim 4, with a sample from a subject.
